(19) United States
(12) Patent Application Publication
(10) Pub. No.: US 2016/0263324 A1
(43) Pub. Date: Sep. 15, 2016

(54) PORTABLE MEDICINE INJECTION DEVICE

(71) Applicant: YOFIMETER, LLC, La Jolla, CA (US)

(72) Inventors: Gad Shaanan, La Jolla, CA (US); Marc Goldman, San Diego, CA (US); Bruce Busby, San Diego, CA (US); Aleks Manulis, San Diego, CA (US)

(21) Appl. No.: 15/068,244

(22) Filed: Mar. 11, 2016

Related U.S. Application Data
(60) Provisional application No. 62/133,144, filed on Mar. 13, 2015.

(51) Int. Cl. A61M 5/20; A61M 5/315

(52) U.S. Cl. CPC A61M 5/20; A61M 5/31593; A61M 5/31585; A61M 2005/2026; A61M 2005/2093; A61M 2005/3114

(57) ABSTRACT

A medicine dispensing device can include a recess configured to receive a medicine cartridge having a plunger assembly and a medicine chamber, and a gear assembly configured to cause displacement of the plunger assembly to dispense medicine or draw medicine into the chamber. The gear assembly can be activated by both a motorized and manual activation mechanism.

INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims priority to U.S. Provisional Patent Application Ser. No. 62/133114 filed on Mar. 13, 2015 and entitled “PORTABLE MEDICINE INJECTION DEVICE”, the content of which is hereby incorporated by reference in its entirety.

### BACKGROUND OF THE INVENTION

1. Field of the Invention

The present invention relates to medicine delivery devices and, more particularly, to electronic injection devices.

2. Description of the Related Art

Diabetes mellitus is a group of metabolic diseases associated with high blood sugar, e.g., which may be due to insufficient production of insulin by the body or inadequate response by cells to the insulin that is produced. There are three main types of diabetes mellitus (diabetes). Type 1 diabetes results from the body's failure to produce insulin, and presently requires the person to inject insulin (e.g., manually or using a wearable insulin pump). Type 2 diabetes results from insulin resistance, in which cells fail to use insulin properly, sometimes combined with an absolute insulin deficiency. Types 1 and 2 diabetes are considered chronic conditions that cannot be cured. The third main form, referred to as gestational diabetes, can occur when pregnant women without a previous history of diabetes develop a high blood glucose level, e.g., which can develop into type 2 diabetes, but often resolves after the pregnancy. Other forms of diabetes include congenital diabetes (e.g., due to genetic defects of insulin secretion), cystic fibrosis-related diabetes, steroid diabetes (e.g., due to high doses of glucocorticoids), and other forms of monogenic diabetes.

For example, diabetes, without proper treatment, may cause acute complications, e.g., including hypoglycemia, diabetic ketoacidosis, or nonketotic hyperosmolar coma, or in some instances, may cause serious long-term complications, e.g., cardiovascular disease, chronic renal failure, and/or diabetic retinopathy (retinal damage). Adequate treatment of diabetes is thus important, as well as controlling blood pressure and managing lifestyle factors such as nonsmoking and healthy body weight. Insulin is used to treat the many of the forms of diabetes, including type 1 diabetes. Other medications are used to treat type 2 diabetes. There is a need for improved medicine injection devices for injecting insulin and other medications.

### SUMMARY OF THE INVENTION

Aspects of the present invention include systems, devices, and methods for dispensing medicine using an electronic medicine injection device.

One aspect is a medicine dispensing device. The medicine dispensing device includes a recess configured to receive a removable medicine cartridge, a gear assembly, a first slidable member coupled to the gear assembly and including a medicine cartridge plunger coupling member, wherein rotation of the gear assembly is configured to cause longitudinal movement of the first slidable member within the recess, and a motor coupled to the gear assembly, where activation of the motor is configured to cause rotation of the gear assembly.

In some embodiments, the first slidable member includes an internally threaded shaft and the medicine dispensing device further includes an elongated screw member threadably engaged with the internally threaded shaft, the elongated screw member coupled to the gear assembly such that rotation of the gear assembly causes rotation of the elongated screw member, thereby causing longitudinal movement of the first slidable member.

In some embodiments, the medicine cartridge plunger coupling member includes a slot configured to receive a T-shaped portion of a medicine cartridge plunger assembly.

In some embodiments, the slot is configured to allow the T-shaped portion of a medicine cartridge plunger assembly to rotate within the slot between a locked and unlocked position, wherein when in the locked position, retraction of the first slidable member retracts the medicine cartridge plunger assembly, and when in the unlocked position, retraction of the first slidable member disengages from and does not retract the medicine cartridge plunger assembly.

In some embodiments, the medicine dispensing device includes a manual dispensing actuator having an engaged and an unengaged position, wherein when in the engaged position, movement of the manual dispensing actuator causes rotation of the gear assembly without activation of the motor, and when in the unengaged position, the manual dispensing actuator is not coupled to the gear assembly.

In some embodiments, the manual dispensing actuator is biased in the unengaged position.

In some embodiments, the manual dispensing actuator can be moved to the engaged position without operating any other actuator.

In some embodiments, the manual dispensing actuator includes a second slidable member

In some embodiments, the a first position of the second slidable member is the unengaged position, and the second slidable member is configured to slide to a second position that is the engaged position, and further sliding of the second slidable member causes rotation of the gear assembly.

In some embodiments, the medicine dispensing device includes a rotatable counter wheel having a plurality of numbers inscribed thereon and a counter wheel opening, wherein the counter wheel opening is configured to provide visibility of one of the plurality of numbers.

In some embodiments, the counter wheel is coupled to the manual dispensing actuator, wherein actuation of the manual dispensing actuator causes one or more discrete amounts of medicine to be dispensed and causes rotation of the counter wheel such that a number visible in the counter wheel opening is indicative of the number of the one or more discrete amounts of medicine dispensed.

In some embodiments, the gear assembly includes a manual drive gear and one or more motorized drive gears, the manual drive gear is coupled to one of the motorized drive gears, wherein when in the engaged position, movement of the manual dispensing actuator causes rotation of the manual drive gear, wherein rotation of the manual drive gear causes rotation of the motorized drive gears, wherein rotation of the motorized drive gears causes longitudinal movement of the first slidable member.

In some embodiments, the motor is configured to rotate the gear assembly in a forward direction and a reverse direction, wherein rotation of the gear assembly in the forward direction causes the first slidable member to advance into the recess, and wherein rotation of the gear assembly in the reverse direction causes the first slidable member to retract out of the recess.

In some embodiments, rotation of the gear assembly in the forward direction causes the medicine cartridge plunger assembly to advance towards a dispensing end of the medicine cartridge, wherein advancement of the plunger assembly towards the dispensing end of the medicine cartridge causes fluid within the between the medicine cartridge plunger assembly and the dispensing end of the medicine cartridge to be dispensed from the medicine cartridge, wherein rotation of the gear assembly in the reverse direction causes the medicine cartridge plunger assembly to retract away from the dispensing end of the medicine cartridge, wherein retraction of the medicine cartridge plunger assembly away from the dispensing end of the medicine cartridge causes the dispensing end of the medicine cartridge to draw fluid in to the medicine cartridge.

In some embodiments, the medicine dispensing device includes a user interface, the user interface including a display configured to display data related to the dispensing of medicine from the medicine device, wherein the user interface is further configured to receive a user selection of an amount of medicine; and

a processor configured to receive data from the user interface, wherein the processor is configured to instruct the motor to drive the gear assembly.

In some embodiments, the medicine dispensing device includes an electrical power source.

In some embodiments, the medicine dispensing device includes one or more sensors configured to measure longitudinal displacement of the first slidable member.

In some embodiments, the one or more sensors include one or more of an electronic caliper, a magnetic sensor, a timer, a barcode scanner or an RF reader.

In some embodiments, the medicine dispensing device includes a communication module, wherein the communication module is configured to transmit data to an external device.

In some embodiments, the external device is an analyte monitoring device.

In some embodiments, the user interface is configured to display one or more of the time, battery status, wireless communication status, time of previous medicine dispensing data, amount of medicine in the vial, and recommended dose amount.

In some embodiments, the medicine dispensing device includes one or more sensors configured to detect a position of the medicine cartridge within the recess, one or more sensors configured to detect an injection event; and a processor configured to limit a number of injection events that can be performed when the medicine cartridge is in an injection position.

In some embodiments, in response to a detection that the medicine cartridge has moved away from an injection position, the processor is configured to retract the first slidable member, wherein the first slidable member disengages from and does not retract the medicine cartridge plunger assembly.

In some embodiments, the medicine dispensing device includes a removable medicine cartridge within the recess, the removable medicine cartridge including, a chamber configured to store medicine therein, a plunger assembly positioned within the chamber, configured to move longitudinally within the chamber, wherein the plunger assembly is coupled to the medicine cartridge plunger coupling member, and a septum configured to receive a needle.

In some embodiments, the plunger assembly includes a plunger seal and a plunger insert, wherein the plunger insert is coupled to the medicine cartridge plunger coupling member.

In some embodiments, the plunger insert includes a T-shaped portion coupled to the medicine cartridge plunger coupling member.

In some embodiments, the removable medicine cartridge includes a tab extending from an exterior wall of the removable medicine cartridge, wherein the tab is configured to allow for rotation of the removable medicine cartridge within the recess, wherein rotation of the removable medicine cartridge within the recess allows for coupling and uncoupling of the removable medicine cartridge with the first sliding member.

In some embodiments, the medicine dispensing device includes a switch extending at least partially into the recess configured to detect the presence of the cartridge within the recess.

In some embodiments, the medicine dispensing device includes a motorized dispensing actuator, wherein the motorized dispensing actuator is configured to actuate the motor while an external pressure is being applied to the motorized dispensing actuator.

Another aspect is a kit. The kit includes a medicine dispensing device and a medicine filling tool, wherein the medicine filling tool includes an adapter member configured to couple to an end of the medicine dispensing device, and a hollow cylinder coupled to the adapter member and configured to receive a medicine vial, wherein the filling tool is shaped such that placement of a medicine vial within the hollow cylinder aligns the medicine vial with the recess and any medicine cartridge therein.

Another aspect is a method for operating a medicine dispensing device. The method includes inserting a medicine cartridge into a recess, the medicine cartridge including a chamber configured to store medicine therein, a plunger assembly positioned within the chamber, configured to be moveable longitudinally within the chamber, and a septum at one end of the chamber, inserting one end of a needle assembly into the septum, inserting an opposite end of the needle assembly into a medicine vial containing medicine, drawing medicine from the medicine vial through the needle assembly into the chamber of the medicine cartridge, and dispensing medicine from the medicine cartridge through the needle.

In some embodiments, the method includes activating the motor to perform one or more of drawing medicine from the medicine vial and dispensing medicine from the medicine cartridge.

In some embodiments, the method includes programming one or more of an amount of medicine to draw from the medicine vial and an amount of medicine to dispense from the medicine cartridge using a user interface.

In some embodiments, the method includes detecting, via one or more sensors, the occurrence of an injection event.

In some embodiments, the method includes displaying data on a user interface.

In some embodiments, the method includes transmitting data to an external device.

In some embodiments, the method includes positioning the medicine cartridge into an injection position, wherein the medicine dispensing device is configured so that an injection event is prevented if the medicine cartridge is not in the injection position, detecting, via one or more sensors, the presence of the medicine cartridge in the injection position, detecting, via one or more sensors, the occurrence of one or more injection events, and limiting the number of injection events that can occur when the medicine cartridge is in the injection position.

In some embodiments, the method includes coupling the plunger assembly of the medicine cartridge with the plunger coupling member of the medicine dispensing device, wherein dispensing medicine includes advancing the plunger assembly within the interior of the medicine cartridge, wherein the advancing the plunger assembly includes advancing the plunger coupling member of the medicine dispensing device, and moving the medicine cartridge out of the injection position, wherein in response to moving the medicine cartridge out of the injection position, the plunger coupling member uncouples from the plunger assembly and automatically retracts without retracting the plunger assembly.

Another aspect is a medicine cartridge for use in a medicine dispensing device. The medicine cartridge includes a chamber configured to store medicine therein, a plunger assembly positioned within the chamber, configured to move longitudinally within the chamber, wherein the plunger assembly includes a plunger seal and a plunger insert, wherein the plunger insert is configured to couple with a medicine cartridge plunger coupling member of a medicine dispensing device, a tab extending from an exterior wall of the medicine cartridge, wherein the tab is configured to allow for rotation of the removable medicine cartridge within a recess of a medicine dispensing device, wherein rotation of the removable medicine cartridge within the recess allows for coupling and uncoupling of the removable medicine cartridge with the medicine cartridge plunger coupling member, a ridge extending from the exterior wall of the medicine cartridge, wherein the ridge is positioned to allow for activation of a switch in the interior of the medicine dispensing device, and a septum configured to receive a needle.

Another aspect is a method for operating a medicine dispensing device. The method includes inserting a medicine cartridge into a recess of the medicine dispensing device, the medicine cartridge including a chamber configured to store medicine therein and a plunger assembly positioned within the chamber, configured to be moveable longitudinally within the chamber, securing a catheter to one end of the medicine cartridge, activating a motor to dispense medicine from the medicine cartridge and dispensing medicine from the medicine cartridge through the catheter.

In some embodiments, the method includes programming one or more of an amount of medicine to dispense from the medicine cartridge through the catheter and a length of time over which to dispense the amount of medicine using a user interface.

### BRIEF DESCRIPTION OF THE DRAWINGS

FIG. 1 depicts a perspective view of an illustrative embodiment of an injection device configured for dispensing medicine.

FIG. 2 depicts a perspective view of an illustrative embodiment of an injection device.

FIG. 3 depicts a perspective view of an illustrative embodiment an injection device.

FIG. 4A depicts a perspective view of an illustrative embodiment of a medicine cartridge.

FIG. 4B depicts a cross-sectional view of an embodiment of a medicine cartridge.

FIG. 5 depicts a rear view of an illustrative embodiment of an injection device.

FIG. 6 depicts rear view of an illustrative embodiment of an interior of an injection device.

FIG. 7 depicts a cross-sectional view of an illustrative embodiment of an injection device.

FIG. 8A depicts a cross-sectional view of an illustrative embodiment of an injection device.

FIG. 8B depicts a cross-sectional view of an illustrative embodiment of an injection device.

FIG. 8C depicts a cross-sectional view of an illustrative embodiment of an injection device.

FIG. 9A depicts a sectional view of an illustrative embodiment of an injection device.

FIG. 9B depicts a sectional view of an illustrative embodiment of an injection device.

FIG. 9C depicts a sectional view of an illustrative embodiment of an injection device.

FIG. 9D depicts a sectional view of an illustrative embodiment of an injection device.

FIG. 10 depicts a perspective view of an illustrative embodiment of an injection device.

FIG. 11 depicts a schematic view of an illustrative embodiment of an injection device.

FIG. 12 depicts an illustrative embodiment of an injection device having several exterior components removed.

FIG. 13 depicts an illustrative embodiment of an injection device having several exterior components removed.

FIG. 14 depicts an illustrative embodiment of an injection device having several exterior components removed.

FIG. 15 depicts an illustrative embodiment of a counter wheel for use in an injection device.

### DETAILED DESCRIPTION OF THE PREFERRED EMBODIMENT

As will be appreciated by one skilled in the art, there are numerous ways of carrying out the examples, improvements, and arrangements of a medicine delivery device in accordance with embodiments of the invention disclosed herein. Although reference will be made to the illustrative embodiments depicted in the drawings and the following description, these embodiments are not meant to be exhaustive of the various alternative designs and embodiments that are encompassed by the disclosed invention. Those skilled in the art will readily appreciate that various modifications may be made, and various combinations can be made, without departing from the invention.

Some embodiments provide systems, methods, and devices for injecting a discrete amount of medicine using a portable medicine injection device in accordance with an amount input by a user.

In one embodiment, a portable medicine injection device is provided that is configured to administer medicine, such as insulin or any other medications that can be injected into a patient, through a motor driven injection. The portable medicine injection device can include a chamber for receiving a medicine cartridge, a gear assembly, a motor, a lead screw, a slidable manual injection button, a user interface, a processor, an electrical power source, and a communication module. The motor can be engaged to a motorized drive gear of the gear assembly. The gear assembly can be engaged to the lead screw. The lead screw can be coupled to a screw nut including a shaft and a plunger coupling member configured to couple with a plunger assembly of the medicine cartridge. Activation of the motor can cause rotation of the gear assembly. Rotation of the gear assembly can cause rotation of the lead screw. Rotation of the lead screw can cause longitudinal displacement of the screw nut, thereby causing displacement of the plunger assembly. Displacement of the plunger assembly within the medicine cartridge can cause medicine to be dispensed from the cartridge or into the cartridge. An amount of medicine dispensed can be selected using the interface of the injection device. The processor can receive data from the user interface and can instruct the motor to drive the gear assembly. The motor may be activated, and the medicine administered, when a button, trigger, or other activation mechanism on the portable medicine injection device is activated. An electronic counter linked to the motor and screw nut movement can control the dispensing. The portable injection device can further include a reverse control to reset the device to accept a new cartridge and allow the device to be used for multiple injections. The device can also be used to fill a cartridge using a reverse feature.

The medicine injection device can further include a manual injection feature that allows for manual injection of medicine. The manual injection feature can include a slidable manual injection button, a counter wheel, and a manual drive gear. The manual injection button can be slid along a track to cause rotation of the manual drive gear. The manual drive gear is part of the gear assembly and coupled to the motorized drive gear such that rotation of the manual drive gear causes rotation of the motorized drive gear.

In another embodiment, a portable medicine injection device can include a chamber for receiving a medicine cartridge, a gear assembly, a motor, a lead screw, a manual activation actuator, a rotatable manual injection knob, a user interface, a processor, an electrical power source, one or more sensors, a pawl, and a communication module. The motor can be engaged to a gear assembly. The gear assembly may include a motor drive gear couple to the motor, and a screw gear coupled to the lead screw. The motor drive gear can selectively engage the screw gear. The lead screw can be coupled to screw nut including a shaft and a plunger coupling member configured to couple with a plunger assembly of the medicine cartridge. The screw nut can include a plunger coupling mechanism to engage a plunger of the vial. Activation of the motor can rotate the motor drive gear. When the motor drive gear is engaged with the screw gear, rotation of the motor drive gear can cause rotation of the screw gear, causing the longitudinal movement of the screw nut. The longitudinal movement of the screw nut can displace the plunger, causing medicine to be dispensed from the cartridge or drawn into the cartridge.

The medicine injection device can further include a manual injection feature that allows for the disengagement of the motor and manual injection of medicine. The manual injection feature can include a manual medicine dispensing knob, a counter wheel, a manual mode activation button, and a reset button. Engaging the manual mode activation button can disengage the motor drive by disengaging the motor drive gear from the screw gear, and can allow for the use of the manual dispensing knob. The manual dispensing knob can be coupled to the screw gear, and can be rotated to dispense medicine. The counter wheel can display the number of units of medicine dispensed. The reset button can be engaged after an injection to reset the counter wheel. Engaging the reset button can also re-engage the motor drive. The counter wheel can engage the manual drive mechanism, the manual mode activation button, and the reset button through a system of gears. The manual injection feature may further include a pawl that engages the dispensing knob to prevent the knob from rotating in the reverse direction during manual operation.

The medicine injection device may also contain a communication module, configured to allow for communication with one or more external devices. The communication module may communicate with external devices through, for example, wireless communication, Bluetooth communication (e.g., BTLE), or cellular communication. The medicine injection device may connect to a personal electronic device such as a smart phone, tablet, or personal computer, or may transmit data to a server. The injection device may communicate information to a patient or to a care provider. The injection device may further be configured to communicate with other medical devices, such as, for example, an analyte measuring device. The analyte measuring device may transmit information relevant to the time and amount of medicine for an injection event. Suitable analyte measuring devices are described, for example, in U.S. Pat. No. 8,333,716, issued on Dec. 18, 2012, U.S. Pat. No. 8,333,717, issued on Dec. 18, 2012, U.S. Pat. No. 8,292,826, issued on Oct. 23, 2012, U.S. Pat. No. 8,961,432, issued on Feb. 24, 2015, U.S. Patent Publication No. 2013/0085349, published on Apr. 4, 2013, U.S. Patent Publication No. 2015/0205930, published on July 23, 2015 and U.S. patent application Ser. No. 14/371,401, filed on Jul. 9, 2014, the content of each being hereby incorporated by reference in their entirety.

The portable medicine injection device may further include a plurality of sensors to determine various states and conditions related to the portable medicine injection device. The portable medicine injection device can include sensors for determining the type of medicine inserted into the device, for example, different types of insulin. The device may detect a code on a vessel containing the medicine, such as a bar code or RFID tag. A bar code scanner or RF reader may be used. The device can also include sensors for determining when an injection occurred and how much medicine was injected. The sensors can include a timer for determining when an injection occurred. The device may use information from the timer to notify a user of a recent injection event if the user tries to inject medicine multiple times in a short time frame. An electronic caliper may be used to determine the amount of medicine injected. In one embodiment, the electronic caliper can measure the longitudinal movement of the shaft of the screw nut. Alternatively, a magnetic sensor may be used to sense the movement of a magnet on a gear of the device (such as the drive gear, screw gear, or counter wheel gear shown below) to determine the amount of medicine injected. The magnetic sensor may be used to measure the longitudinal movement of the shaft of the screw nut. The sensors may further determine the amount of medicine drawn into a vial and the amount of medicine left in a vial following an injection event.

The portable medicine injection device may include a medicine cartridge. The medicine cartridge can be a separate piece configured to engage a portable medicine injection device. The medicine cartridge can be disposable. The cartridge can include a septum for receiving a needle for insertion into a patient. The cartridge can also include a plunger assembly to allow for forward and reverse plunger movements. The plunger assembly can engage the plunger coupling member of the screw nut. The screw nut can drive the plunger assembly in the direction of the needle for injection of medicine into a patient. Additionally, the needle can be inserted into a source of medicine and the screw nut can drive the plunger assembly in the reverse direction to draw medicine into the cartridge.

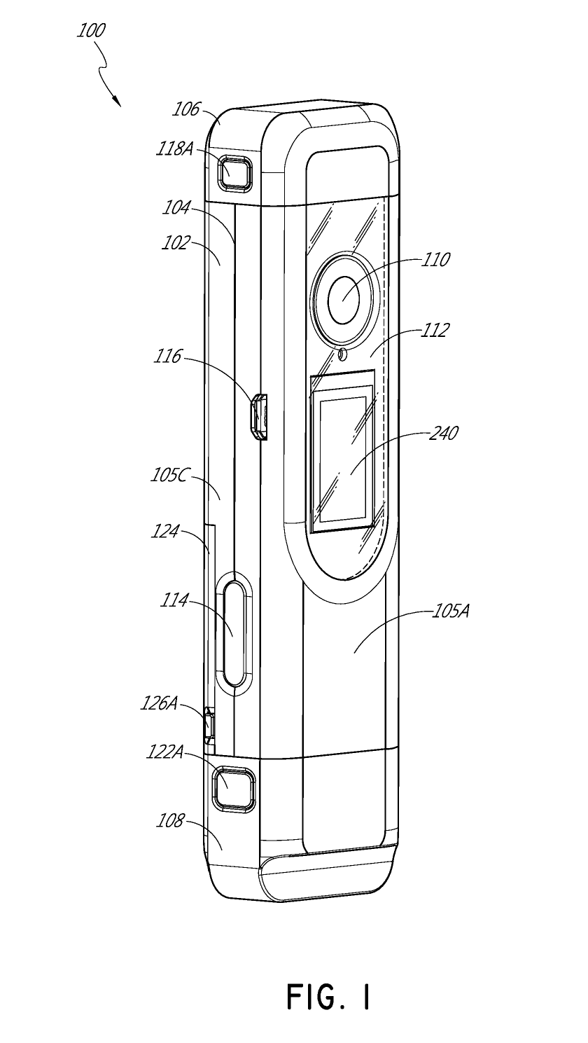

FIG. 1 depicts a perspective view of an illustrative embodiment of an injection device 100 configured for dispensing medicine. The injection device 100 includes a housing 102, a multi-function button 110, a display cover 112, a motorized injection button 114, and a charging port 116. The injection device includes a front 105A, a back (not shown), a first side 105C, and a second side (not shown). The housing 102 includes a cap 106 positioned at a proximal end of the injection device 100, a cap 108 positioned at a distal end of the injection device 100, and a body 104 positioned between the cap 106 and the cap 108.

FIG. 1 shows a release button 118A coupled with the cap 106 on the first side 105C of the injection device 100. The button 118A can be configured to allow for release and removal of the cap 106 from the body 104. In some embodiments, the cap 106 can be coupled with a corresponding release button on the second side of the injection device 100 to allow for release and removal of the cap 106 from the body 104. A release button 122A is shown coupled with the cap 108 on the first side 105C of the injection device 100. The button 122A can be configured to allow for release and removal of the cap 108 from the body 104. In some embodiments, the cap 108 can be coupled with a corresponding release button on the second side of the injection device 100 to allow for release and removal of the cap 108 from the body 104.

The display cover 112 can be positioned on the front 105A of the injection device 100. The display cover 112 can be transparent or semi-transparent to allow for viewing of a display screen 240 positioned beneath the display cover 112. The display screen 240 can be configured to display data to a user. The multi-function button 110 can also be positioned on the front 105A of the injection device 100. The cover 112 can include an opening to allow for access to the multi-function button 110, the multi-function button 110 being positioned therein. The multi-function button 110 can be part of a user interface configured to receive data inputs from a user. For example, the multi-function button 110 can be configured to allow for a selection of an amount of medicine to dispense. The multi-function button 110 may include any user input feature, including, but not limited to, a button, a scroll-wheel, and a keypad. In some embodiments, the multi-function button 110 includes a central selection button and a toggle feature that can make selections in four orthogonal directions.

The injection button 114 can be positioned within the body 102 on the side 105C of the injection device 100. The injection button 114 can be configured to operate a motorized injection mechanism. In some embodiments, the injection button 114 can be configured to operate the motorized injection mechanism to perform an injection event, such as for example, dispensing medicine from the injection device 100 or drawing medicine into the injection device 100 from an external source. The injection button 114 can further be configured to operate the motorized injection mechanism to draw medicine into the injection device 100 from an external source. In some embodiments, the injection button 114 is configured to cause a particular injection event in response to pressing and releasing the injection button 114. In some embodiments, the injection button 114 is configured to cause a particular injection event in response to holding the injection button 114 in a depressed position. While an injection button 114 is described, it should be recognized imitation of an injection event is not limited to buttons, but can be performed using, for example, switches, dials, levers, touch screen interfaces, or any other input devices.

The charging port 116 can be positioned within the body 104 on the side 105C of the injection device 100. The charging port 116 can be configured to receive a charging cable or connector to provide power to the injection device 100 or to charge a battery within the injection device 100. The charging port 116 can be a USB port, a mini-USB port, a micro-USB port, or any other port capable of receiving a charging cable. The charging port 116 can be configured to receive a USB cable, a min-USB cable, a micro-USB cable, or any other charging cable capable of delivering power to an electronic device.

FIG. 1 also shows part of a cover 124 and a cover release button 126A. The cover 124 is extends over the back of the injection device 100 and has portion positioned on the side 105C of the body 104 of the injection device 100. The cover release button 126A is positioned on the body 104 on the side 105C of the injection device 100 and can be configured to allow for release and removal of the cover 124 from the injection device 100.

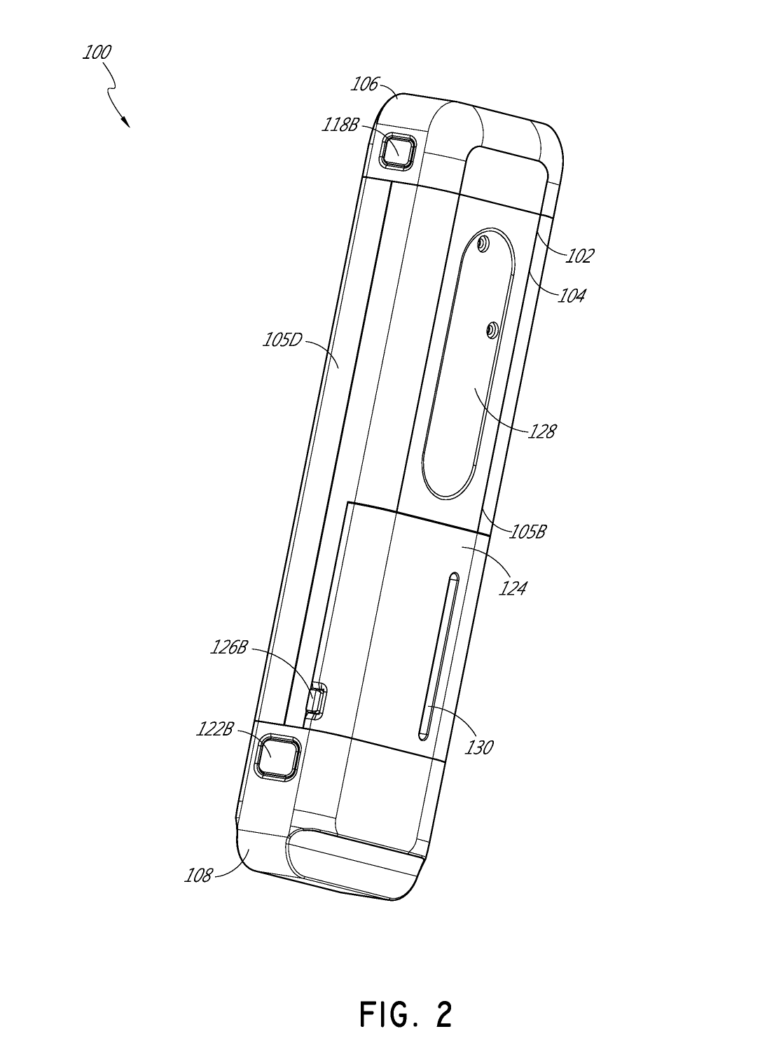

FIG. 2 depicts a perspective view of an illustrative embodiment of the injection device 100 showing a back 105B and a side 105D of the injection device 100. FIG. 2 shows a release button 118B coupled with the cap 106 on the second side 105D of the injection device 100. The button 118B, along with button 118A, can be configured to allow for release and removal of the cap 106 from the body 104. A release button 122B is shown coupled with the cap 108 on the second side 105D of the injection device 100. The button 122B, along with button 122A, can be configured to allow for release and removal of the cap 108 from the body 104. The injection device 100 further includes a cover release button 126B coupled with the cover 124 and positioned on the side 105D of the injection device 100. The cover release button 126B, along with the cover release button 126A can be configured to allow for release and removal of the cover 124 from the body 104. Positioned within the cover 124 is an opening 130 configured to provide a view of a medicine vial housed in the interior of the injection device 100. The body 104 further includes a recess 128 configured to receive and secure a label. A label can include a type of medicine housed within the injection device, such as for example, a type of insulin.

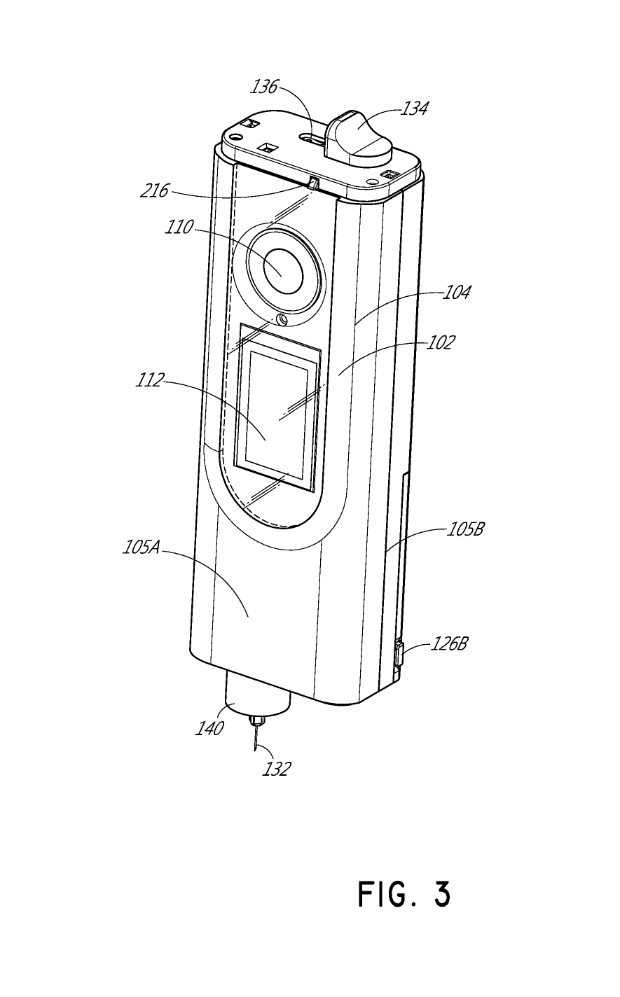

FIG. 3 depicts a perspective view of an illustrative embodiment of the injection device 100 having the cap 106 and cap 108 removed. Protruding from a distal end of the body 104 is a needle 132 configured to deliver medicine to a patient. The needle 132 is positioned within a needle housing 140, which is then coupled to a medicine cartridge. Protruding from the proximal end of the body 104 is a slide button 134 configured to slide within a slot 136. The slide button 134 is part of a manual injection mechanism that allows a user to manually dispense medicine from the needle 132. An arm 216 of a pawl is shown extending out of the injection device 100.

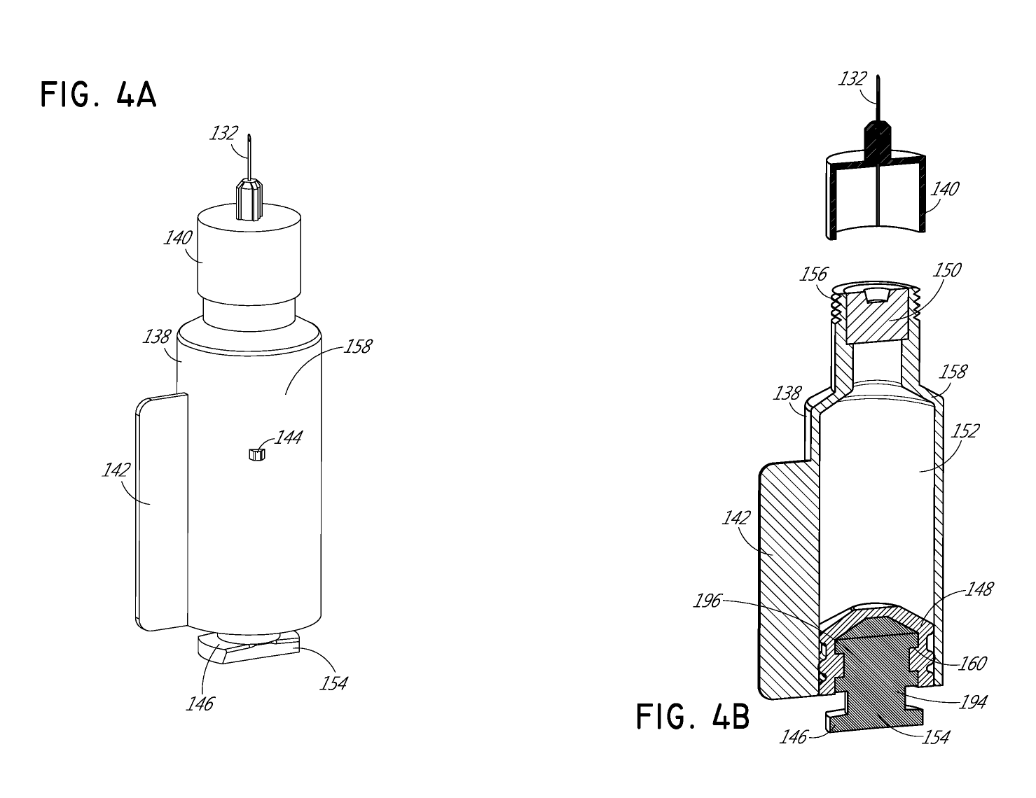

FIG. 4A depicts an embodiment of a medicine cartridge 138 configured to store medicine. The medicine cartridge 138 includes the needle 132, needle housing 140, a body 158, a tab 142, a protrusion 144, and a plunger assembly including a plunger insert 154 and a plunger seal (not shown). The plunger insert 154 includes an elongated section 146 at its proximal end.

FIG. 4B depicts a cross-sectional view of the cartridge 138 showing a medicine chamber 152 configured to receive and store medicine. The needle housing 140 is configured to secure to a connection section 156 at a distal end of the body 158. The connection section 156 may be threaded and configured to engage a complimentary threaded section of the needle housing 140. A septum 150 is located within the interior of the distal end of the body 158. The septum is configured to receive the needle 132 therethrough and to provide a seal around the needle 132 so that medicine does not leak out of the cartridge 138 following insertion of the needle 132. A plunger seal 148 is positioned within the interior of the chamber 148 proximal to the septum 150. A proximal end of the plunger seal 148 includes a receptacle 160 configured to receive and securably engage a distal end of the plunger insert 154. Immediately distal to the elongated section 146 is a cylindrical shaft section 194 having a diameter smaller than a length of the elongated section 146. The plunger insert 154 includes a connection section 196 positioned distally to the cylindrical shaft section 194. The connection section 196 is shaped and configured to securably engage the plunger seal 148. The diameter of the cylindrical shaft section 194 is less than a length or circumference of a portion of the connection section 196. The plunger insert 154 and plunger seal 148 may both comprise flexible polymers. The plunger seal 148 and/or plunger insert 154 may form a seal at the proximal end of the medicine chamber 152 to prevent medicine from leaking out of the proximal end of the medicine chamber 152.

In an alternative embodiment, the cartridge 138 can be configured to engage a catheter or cannula for use in an infusion pumping operation. For example, the connection section 156 at the distal end of the body 158 can be configured to securably engage a first end of a catheter. A second end of the catheter can be attached via a connector to a cannula for insertion into the subcutaneous tissue of a patient. In some embodiments, an insertion device may be used for insertion of the cannula into the tissue of a patient. In some embodiments, the cartridge 138 can be configured to couple to a commercially available infusion set, such as for example, an Animas® Inset® Infusion Set.

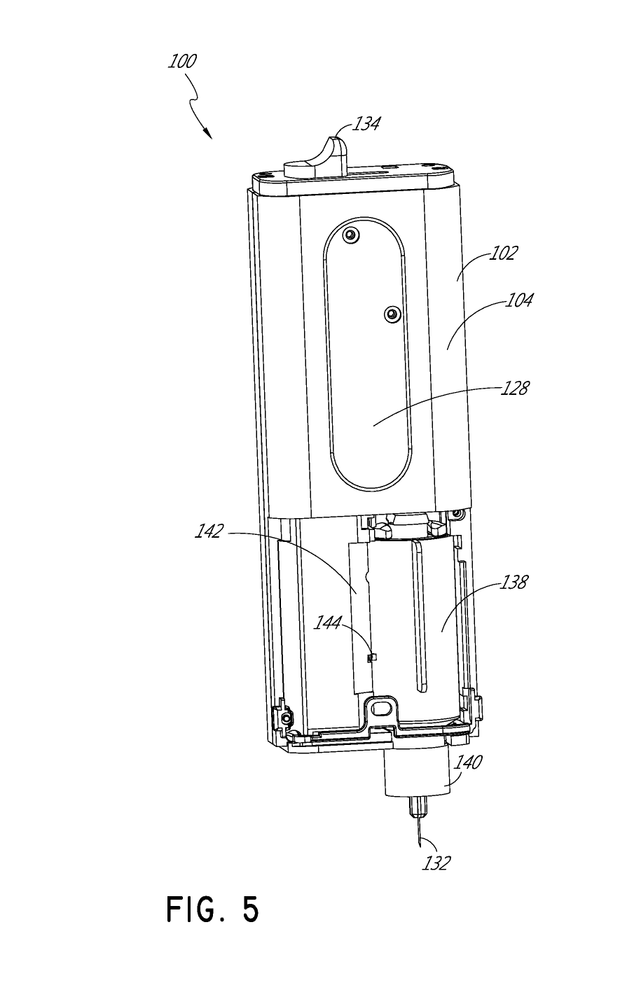

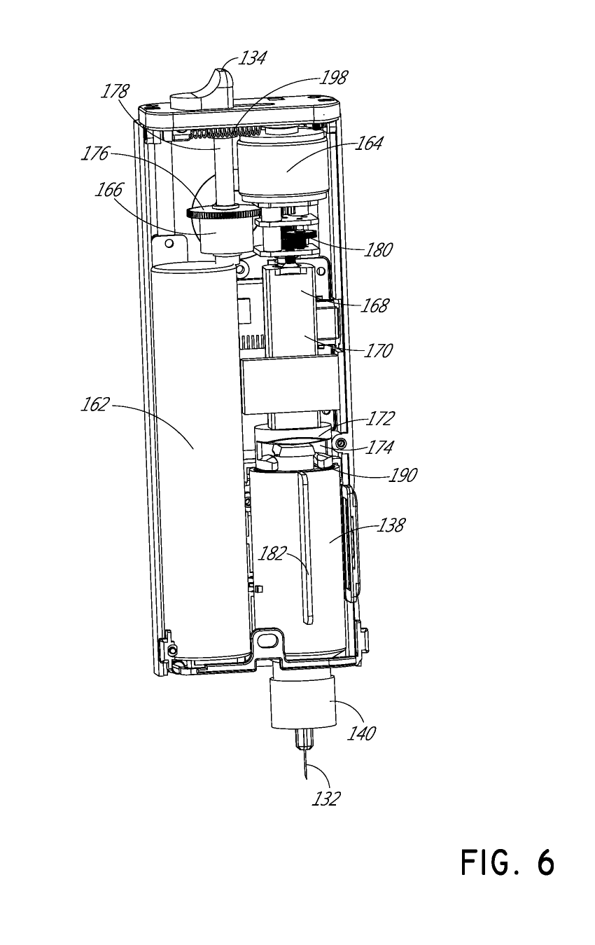

FIG. 5 depicts a rear view of an illustrative embodiment of the injection device 100 having the cap 106, the cap 108, and the cover 124 removed. FIG. 5 shows the medicine cartridge 138 positioned within the body 104. The cartridge 138 can be inserted into and removed from the injection device 100 when the cover 124 is removed.

FIG. 6 depicts a rear view of the injection device 100 with a section of the body 104 removed from the rear 105B of the injection device 100, showing internal components of the injection device 100. The injection device 100 includes a battery 162, a motor 164, a gear assembly 166 and a screw nut 168.

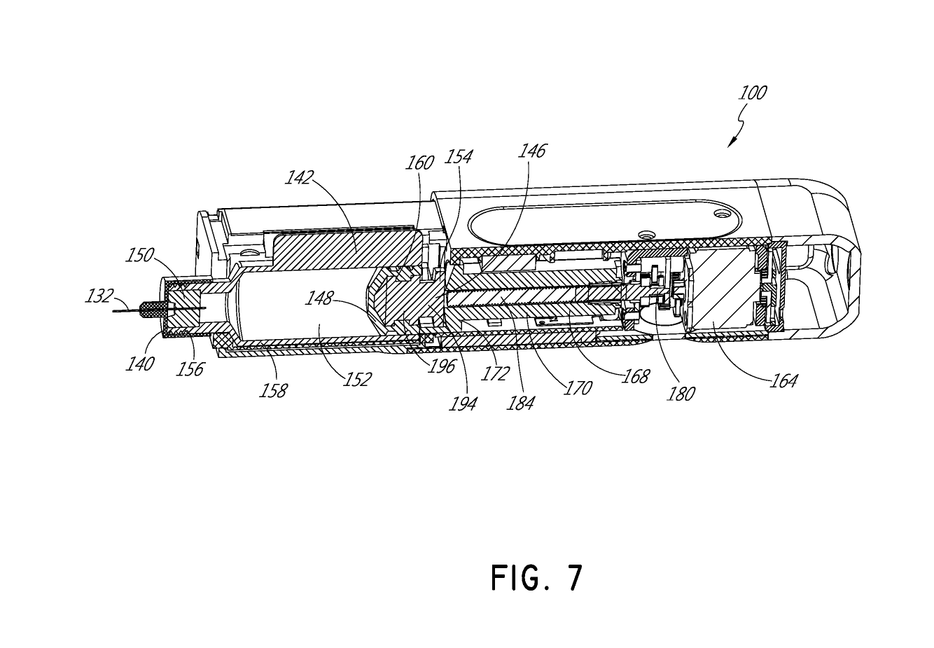

The screw nut 168 includes a shaft 170 and a plunger coupling member 172. The plunger coupling member 172 includes a slot 174. The slot 174 is configured to receive the elongated or T-shaped section 146 of the plunger insert 154. A bottom plate 190 of the plunger coupling member 172 defines the bottom edge of slot 174. FIG. 7 depicts a cross-sectional view of the injection device 100. As shown in FIG. 7, a lead screw 184 can be positioned within the interior of the screw nut 168. The interior of the screw nut 168 can be threaded to threadably engage the lead screw 184.

Referring to FIGS. 6 and 7, The gear assembly 166 includes a manual drive gear 176 and plurality of motorized drive gears 180. The manual drive gear 176 and motorized drive gears 180 are configured to allow for both a motorized a manual injection mechanism.

### Motorized Injection Mechanism

The motorized drive gears 180 are coupled to the motor 164. Activation of the motor 164 can cause rotation of the motorized drive gears 180. The motorized drive gears 180 are coupled to a lead screw (not shown) housed within the shaft 170 of the screw nut 168. Rotation of the motorized gears 180 can cause rotation of the lead screw. The threads on the lead screw engage with threads on the interior of the screw nut 168. The distal surface of the bottom plate 190 is configured to engage the proximal end of the plunger seal 148. A proximal interior surface of the plunger coupling member 172 can engage a proximal surface of the elongated section 146 of the plunger insert 154. Rotation of the lead screw can cause the screw nut 168 to advance towards the distal end of the injection device 100 or retract towards the proximal end of the injection device 100. Advancement of the screw nut 168 can impose a force on the proximal end of the plunger seal 148 and/or insert 154, causing the plunger seal 148 and/or insert 154 to advance within the medicine chamber 152 towards the distal end of the injection device 100. Advancement of the plunger seal 148 within the medicine chamber 152 towards the distal end of the injection device 100 can cause medicine to flow out of the needle 132. The cartridge 138 includes a slot 182 to allow for visibility of the amount of medicine within the chamber 152. In order to draw medicine into the medicine chamber 152, the motor 164 can cause the motorized gears 180 to rotate in a direction opposite the direction of rotation for dispensing medicine, causing the screw nut 168 to retract longitudinally towards the proximal end of the injection device 100. Proximal retraction of the screw nut 168 can cause a force to be applied to the distal surface of the elongated section 146 by a proximal surface of the plate 190, causing the elongated section 146 and plunger seal 148 to advance within the medicine chamber 152 towards the proximal end of the injection device 100. Advancement of the plunger seal 148 towards the proximal end of the injection device 100 can cause medicine to be drawn into the medicine chamber 152.

In alternative embodiments, the injection device 100 can be configured to perform a continuous infusion. As described above, the cartridge 138 can be coupled to a catheter connected to a cannula inserted into a patient. The motor 164 can be activated as described above to deliver medicine from the cartridge 138 through the cannula over an extending period of time, such as for example, several hours or days.

### Medicine Cartridge and Use Thereof

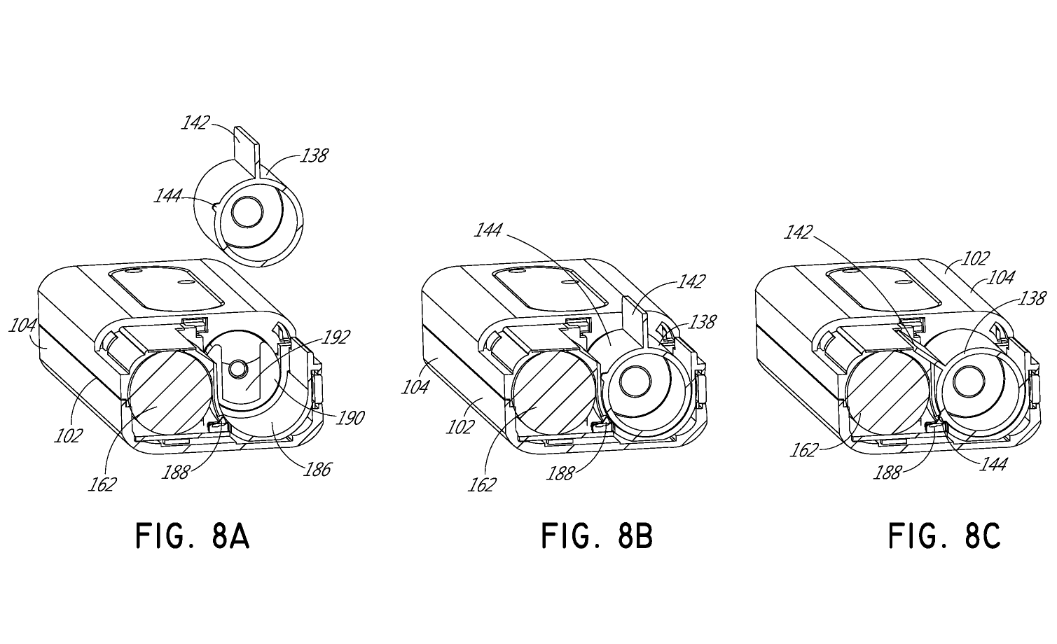

FIG. 8A depicts a cross-sectional view of the injection device 100 showing the cartridge 138 separated from the injection device 100. The injection device 100 includes a receptacle 186 configured to receive the cartridge 138. A microswitch 188 extends into the recess 186. The bottom plate 190 includes a generally u-shaped opening 192, the opening 192 being shaped and configured to allow for securement of the cartridge 138 to the injection device 100. The opening 192 can be configured to receive the cylindrical shaft section 194 of the insert 154 such that the elongated or T-shaped section 146 of the insert 154 is positioned within the slot 174 of the plunger coupling member 172.

FIG. 8B depicts a cross-sectional view of the injection device 100 showing the cartridge 138 positioned within the recess 186. The cartridge 138 is shown in an unlocked position. In the unlocked position, the long axis of the elongated section 146 is positioned so as to be generally aligned with the long axis of the opening 192 such that the elongated section 146 can pass through the opening 192 of the cartridge 138 is advanced in the direction of the distal end of the injection device 100.

FIG. 8C depicts a cross-sectional view of the injection device 100 showing the cartridge 138 positioned within the recess 186. The cartridge 138 has been rotated approximately 45° counterclockwise from the position shown in FIG. 8B to a locked position. In the locked position, the protrusion 144 has engaged the microswitch 188. Rotation of the cartridge 138 causes rotation of the elongated or T-shaped section 146, such that the long axis of elongated section 146 no longer aligns with the long axis of the opening 190, such that distal movement of the plunger assembly and the cartridge 138 within the recess 186 is prevented. In some embodiments, the injection device 100 is configured not to perform a motorized injection if the microswitch 188 is not engaged. When in the locked position, in order to remove the cartridge 138, the cartridge must be rotated in a clockwise direction so that the long axis of the elongated or T-shaped section 146 is aligned with the long axis of the opening 190 to allow for the cartridge to advance distally through the opening 190. Clockwise rotation of the cartridge 138 from locked position causes the protrusion 144 to disengage the microswitch 188. Disengagement of the microswitch 188 may cause automatic retraction of the lead screw 184, and consequently the screw nut 168, to a pre-injection position wherein the lead screw 184 and screw nut 168 are positioned proximally to the proximal end of the medicine chamber 152. In this case, since the elongated or T-shaped section 146 is aligned with the long axis of the opening 190, retraction of the lead screw 184 does not also cause retraction of the plunger assembly, leaving it recessed within the cartridge chamber. This feature prevents a user from re-using a cartridge after it has been removed. In some embodiments, the lead screw 184, and consequently the screw nut 168, are configured not to reverse unless the microswitch 188 is disengaged.

In some embodiments, a processor within the injection device 100 is programmed to allow a predefined series of events to occur for a medicine cartridge 138 after insertion into the injection device 100 based on one or more readings from the microswitch 188. After the protrusion 144 engages the microswitch 188, the processor may allow for a first event in which the plunger seal 148 and plunger insert 154 are advanced towards the distal end of the injection device 100 in order to dispense air from the medicine cartridge 138 into a medicine chamber of an external medicine source. The processor may then allow for a second event in which the plunger seal 148 and insert 154 are retracted towards the proximal end of the injection device 100 in order to draw medicine into the medicine chamber 152. The processor may further allow for a third event in which the plunger seal 148 and plunger insert 154 are advanced towards the distal end of the injection device 100 in order to dispense medicine. The processor may be configured to prevent further events from occurring prior to disengagement of the protrusion 144 from the microswitch 188. In some embodiments, upon disengagement of the injection device, the plunger insert 154 is aligned with the opening 192, and the screw nut 168 retracts such that the insert 154 and plunger seal 148 remain in a bottom portion of the medicine chamber 152. This series of steps can function to prevent re-use of a single medicine cartridge. Re-use of a medicine cartridge can lead to safety issues, such as a broken or weakened cartridge, mixing of medicine with residue from a previous injection, contamination, etc.

### Manual Injection Mechanism

With reference to FIGS. 6 and 7, the manual drive gear 176 is coupled to a gear shaft 178 through a roller clutch. The gear shaft 178 is coupled to the sliding button 134. Movement of the sliding button 134 can cause rotation of the gear shaft 178. The sliding button 134 is further connected to a spring 198 configured to return the sliding button 134 to a starting position following release of the sliding button 134. Rotation of the gear shaft 178 can cause rotation of the manual drive gear 176. The manual drive gear 176 is also coupled to the plurality of the motorized drive gears 180. Rotation of the manual drive gear 176 can cause rotation of the motorized drive gears 180. Rotation of the motorized drive gears 180 can cause medicine to be dispensed as described with respect to the motorized injection mechanism.

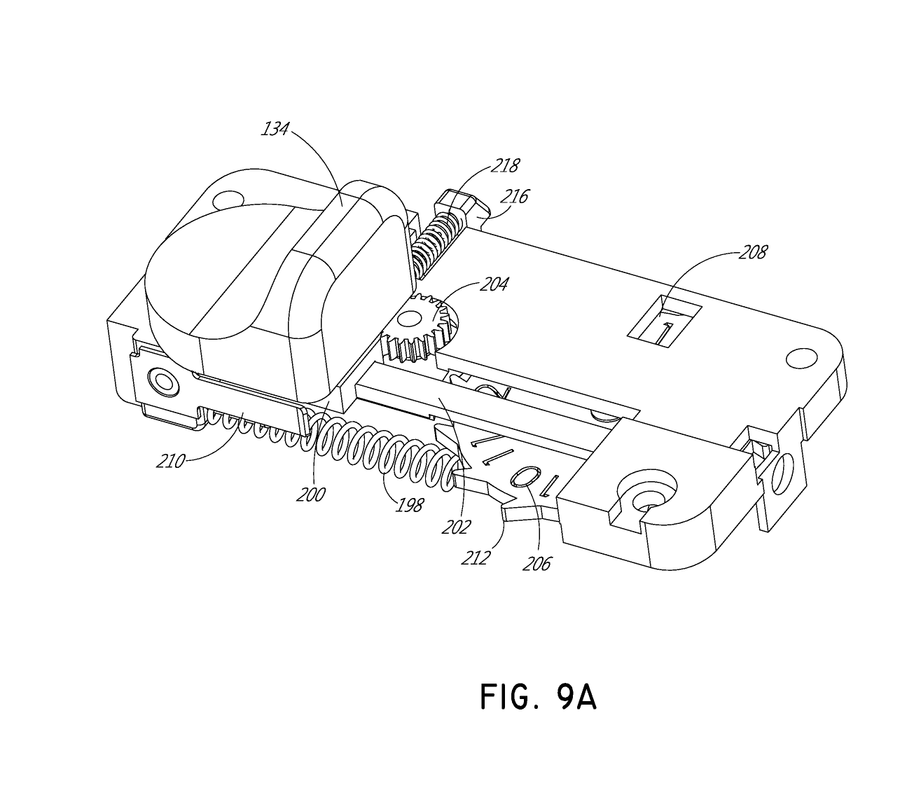

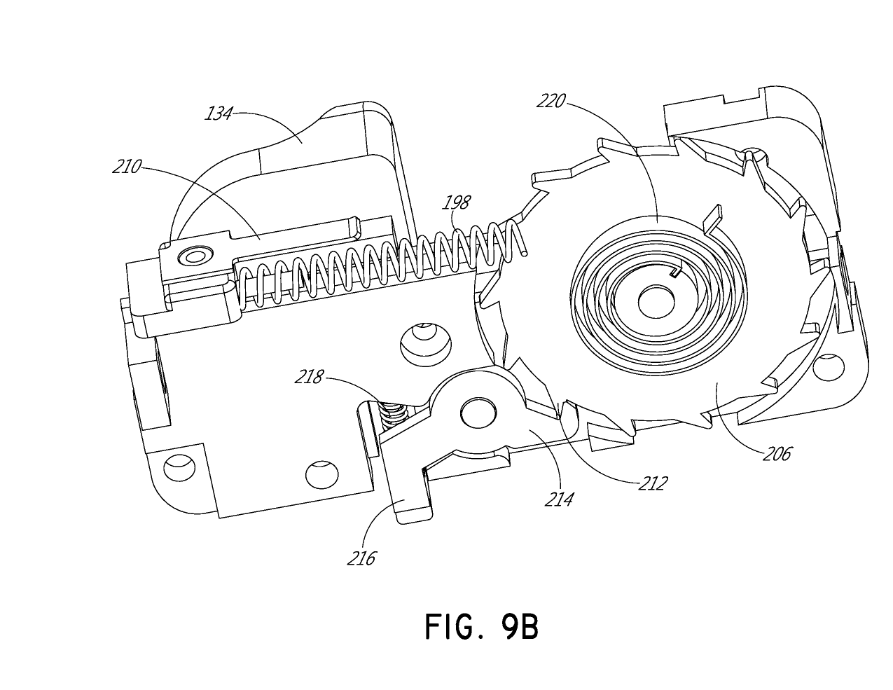

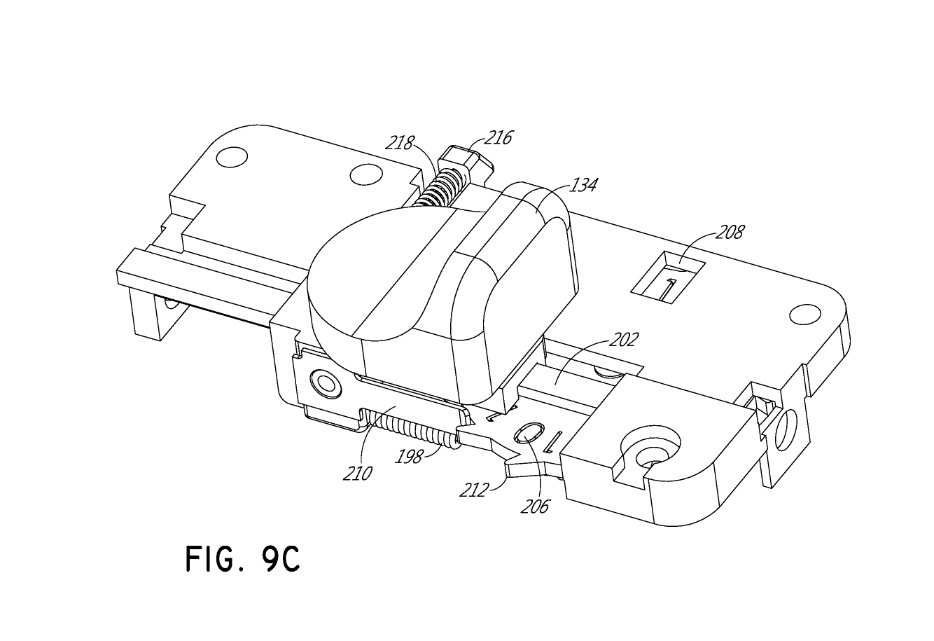

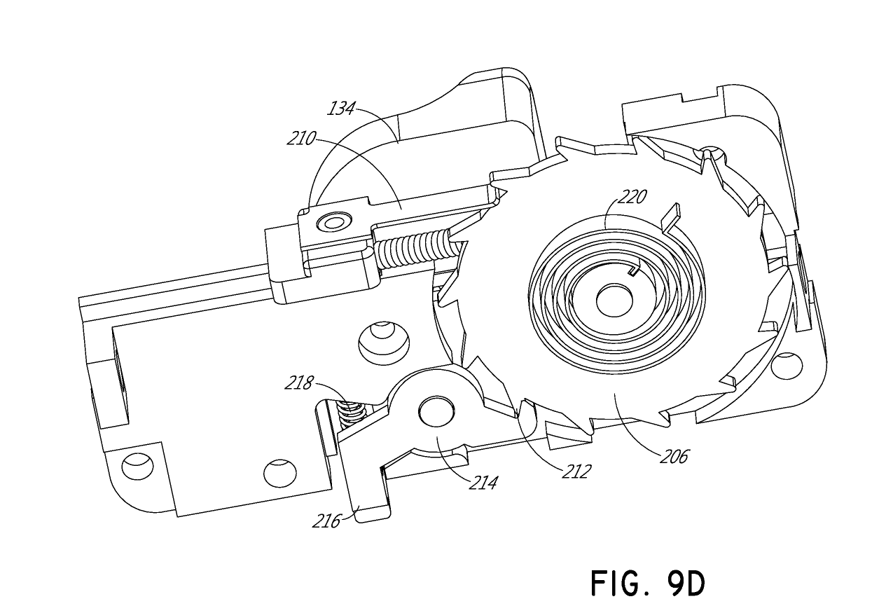

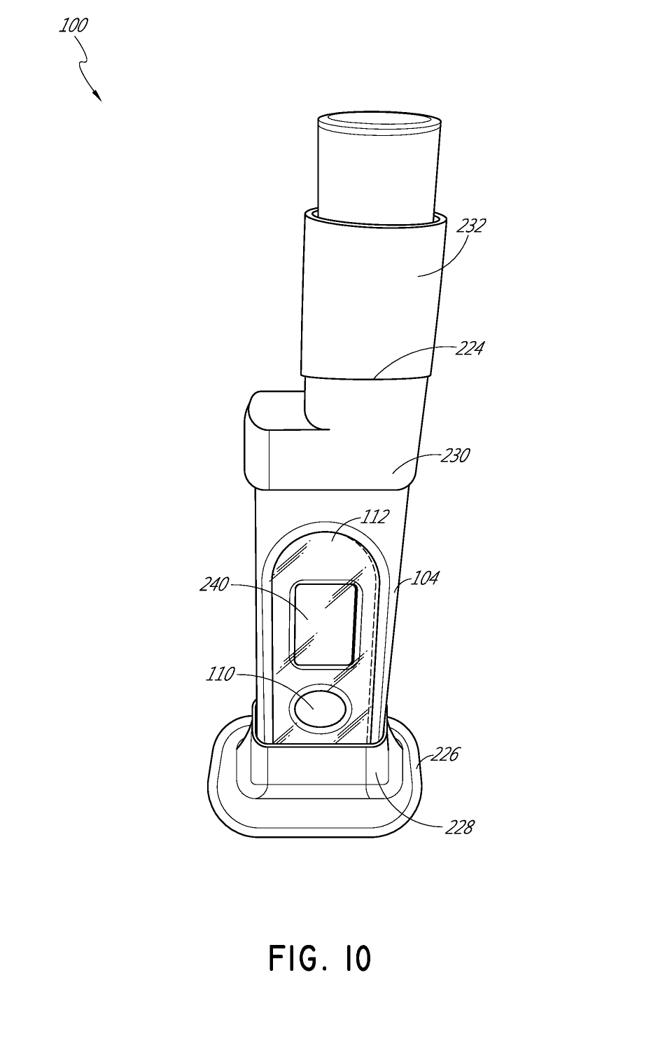

FIG. 9A depicts section view of the injection device 100 showing a sliding button mechanism for manual injection. The sliding mechanism includes the sliding button 134, a rack 200, a track 202, a pinon 204, a counter wheel 206, a counter wheel opening 208, a spring 218, a spring 198 and a pawl 214. In FIG. 9A, the sliding button 134 is shown in a starting position. The sliding button 134 is coupled to the rack 200. The rack 200 is configured to slide along the track 202 when the sliding button 134 is advanced from its starting position towards the counter wheel 206. The rack 200 includes a plurality of teeth on an external surface facing the pinion 204. The plurality of teeth of the rack 200 are configured to engage with complementary teeth on the exterior of the pinion 204. The pinion 204 is engaged to the shaft 170 (shown in FIG. 6) of the injection device 100. When in the starting position, the teeth of the rack 200 do not engage the pinion 204. Advancement of the sliding button 134 causes the rack 200 to advance along track 202. Advancement of the rack 200 along track 202 causes the teeth of the rack 200 to engage the pinion 204. Further advancement of the rack 200 causes the pinion 204 to rotate, thereby causing the shaft 170 to rotate. As previously explained, rotation of the shaft 170 can cause rotation of the manual drive gear 176, and consequently, can cause an injection event. The spring 198 is configured to return the sliding button 134 to a starting position following release of the sliding button 134.

The counter wheel 206 includes a plurality of teeth 212. The top surface of the counter wheel 206 includes a plurality of numbers, one number for each of the teeth 212. The counter wheel opening 208 is configured to provide visibility to a number on the counter wheel 206 positioned below the opening 208. The sliding button 134 can be coupled with an arm 210. As the sliding button 134 is advanced, the arm 210 is configured to engage the teeth 212 of the counter wheel 206.

FIG. 9B depicts a bottom view of the sliding button mechanism of FIG. 9A. The injection device 100 further includes a pawl 214. The pawl 214 is configured to engage the plurality of teeth 212 of the counter wheel 206. The teeth 206 can be angled such that the pawl 214 prevents rotation of the counter wheel 206 in the counterclockwise direction but allows for clockwise rotation of the counter wheel 206. Clockwise rotation of the counter wheel 206 can be configured to provide for sequentially increasing numbers to appear in the opening 208 as the sliding button 134 is repeatedly actuated.

FIG. 9C depicts the sliding button mechanism of FIG. 9A with the sliding button 134 advanced along the track 202. FIG. 9C shows the arm 210 engaging one of the teeth 212 of the counter wheel 206 due to advancement of the sliding button 134. Further advancement of the sliding button 134 along the track 202 can cause rotation of the counter wheel 206 due to further advancement of the arm 210. FIG. 9D depicts a bottom view of the sliding button mechanism in the position shown in FIG. 9C.

In an illustrative embodiment of the present invention, the sliding button 134 can be advanced from the starting position along a length of the track 202 to dispense one increment of medicine. As described previously, advancement of the sliding button 134 causes rotation of the pinion 204, which through the gear assembly 166 and lead screw 184, causes dispensing of medicine. Advancement of the sliding button 134 also causes the arm 210 to engage one of the teeth 212 such that advancement of the arm 210 causes the clockwise rotation of the counter wheel 206. Advancement of the sliding button 134 along the length of the track 202 can cause the counter wheel 206 to rotate such that the pawl 214 engages a next one of the teeth 212 located counterclockwise from an initial one of the teeth 212 engaged by the counter wheel 206 prior to advancement of the sliding button 134. Such rotation of the counter wheel 206 causes the number on the counter wheel 206 visible in the opening 208 to increase by a single unit. In accordance with an illustrative embodiment, advancement of the sliding button 134 from the starting position along the length of the track causes the dispensing of one increment of medicine and a number visible in the opening 208 to increase by one unit, providing a count of the number of increments of medicine dispensed.

The pawl 214 includes pawl arm 216. Pawl arm 216 is configured such that a force exerted on the pawl arm 216 in the direction of the center of the injection device 100 causes the pawl 214 to disengage from the teeth 212 of the counter wheel 206. When the counter wheel 206 is disengaged from the pawl 214, a torsion spring 220 causes the counter wheel 206 to return to a starting position in which the number visible through the opening 208 is zero. The arm 216 is connected to a spring 218 that biases the arm 216 away from the center of the injection device 100 such that when a force is not applied to the arm 216, the arm 216 is biased such that the pawl 214 engages the teeth 212 of the counter wheel 206. Placement of the cap 106 on the proximal end of the body 104 can cause the arm 216 to advance towards the center of the injection device 100, causing release of the teeth 212 of the counter wheel 206. This allows for the pawl 214 to engage the counter wheel 206 when the cap 106 is removed for manual injection, and allows the pawl 214 to disengage from the counter wheel 206 when the cap 106 is repositioned on the proximal end of the body 104 following manual injection and reset the number visible through the opening 208 to zero.

### Dispensing Device Filling Mechanism

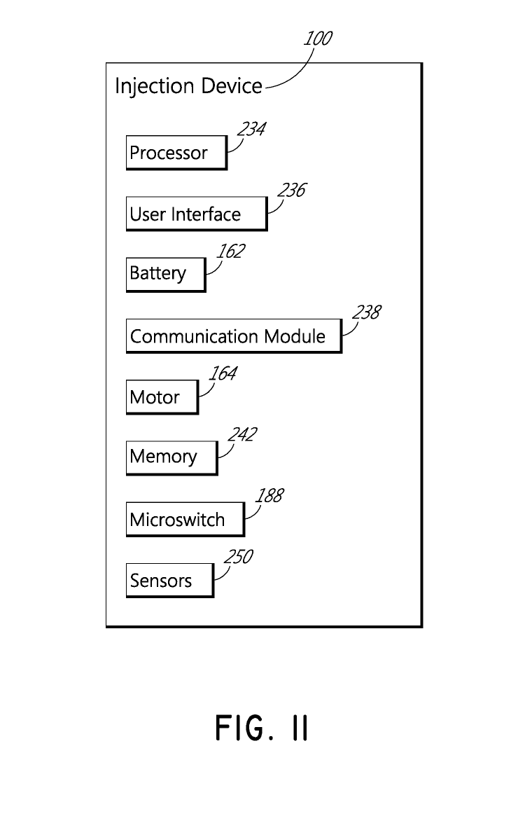

FIG. 10 depicts a perspective view of an illustrative embodiment of an injection device 100 engaged to a base 222 and a filling tool 224. The holder 222 can include a recess 226 configured to receive the proximal end of the injection device 100. The holder 222 can also include a widened section 228 configured to be placed on a flat surface such that, when the injection device 100 is positioned within the holder 222, the long axis of the injection device 100 is substantially perpendicular to the flat surface. The filling tool 224 can include an adapter member 230 configured to be positioned over the distal end of the injection device 100. The filling tool 224 further includes a cylindrical channel 232 configured to receive an external medicine vial. In some embodiments, the injection device 100 can be used to fill the medicine cartridge 138 with medicine. The holder 222 and filling tool 224 can be used to assist in a filling operation. In order to fill the medicine cartridge 138 with medicine, the needle 132 that is coupled to the medicine cartridge 138 can be inserted into an external medicine vial. In some embodiments in which the filling tool 224 is used, the filling tool 224 is first placed over the injection device 100. The medicine vial is inserted into the channel 232 so that the needle 132 pierces a septum or stopper within the external medicine vial to access a medicine chamber within the external medicine vial. Medicine vials, for storing insulin for example, are commonly available in 3 mL, 10 mL, and 20 mL sizes. The filling tool may be shaped and sized to accommodate 3 mL, 10 mL and/or 20 mL medicine vials, or any other commonly used medicine vial size. After the needle 132 is positioned within the medicine chamber of the external medicine vial, the motorized mechanisms can be used to advance the plunger seal 148 towards the distal end of the injection device 100, such that the movement of the plunger seal causes air to be expelled from the medicine chamber 152 and into the external medicine vial. In some embodiments, the filling tool 224 is configured to sufficiently secure the external medicine vial so that the external medicine vial does not disengage from the needle in response to receiving air expelled from the medicine chamber 152. The filling tool 224 can include one or more coupling elements configured to engage with and secure one or more components of the external medicine vial. For example, the filling tool 224 can include one or more ridges configured to engage a lip of the external medical vial. The ridges may be tapered to allow for insertion of the external medical vial into the filling tool 224, but to at least partially restrict movement of the external medical vial out of the filling tool 224. The one or more coupling elements may be positioned in the interior of the adapter member 230 and/or the cylindrical channel 232. Next, the motorized mechanism can be operated in a reverse operation to cause the screw nut 168 to advance towards the proximal end of the injection device 100. Advancement of the screw nut 168 towards the proximal end of the injection device 100 will apply a force on the plunger insert 154 and plunger seal 148, causing the plunger insert 154 and plunger seal 148 to retract away from the needle 132 and towards the proximal end of the injection device 100. Retraction of the plunger seal 148 away from the needle 132 can cause the needle to draw fluid from the medicine chamber of the external medicine vial and into the medicine chamber 152. In some embodiments, the entire filling sequence can be initiated by a single press of the injection button 114.

### Dispensing Device Electronics

FIG. 11 depicts a schematic view of an illustrative embodiment of the injection device 100. The injection device 100 includes a processor 234, a user interface 236, a communication module 238, the battery 162, the motor 164, a memory 242, the microswitch 188, and one or more sensors 250.

The user interface 236 can include the injection button 114, the multi-function button 110, and the display screen 240. The multi-function button 110 can be configured to receive selections for various operating parameters of the injection device 100, selections of data to display on the display screen 240, and/or data or instructions to an external device via the communication module 238. The user interface 236, via the multi-function button 110, can be configured to receive a selection of an amount of medicine to dispense or an amount of medicine to draw into the medicine cartridge 138. The user interface 236 may also be used to select an external device to send data to, to access past medicine dispensing information and/or patient data stored in the memory 242, to set an alarm for dispensing medicine, to input patient data, to input medicine data, and/or to confirm an instruction from the injection device, such as for example, an instruction to inject medicine at a particular time. The user interface 236 can be configured to display one or more of the time, battery status, wireless communication status, time of previous medicine dispensing data, amount of medicine in the cartridge 238, and recommended dose amount. The user interface, via the injection button 114, can be configured to receive an input from a user in order to initiate a filling of the medicine cartridge 138, or motorized dispensing of medicine from the medicine cartridge 138.

The user interface 236 can also be configured to receive a selection of a dosage amount and a length of time over which to deliver the dosage amount. This can allow the injection device 100 to act in a similar manner to an infusion pump in embodiments in which the cartridge 238 is coupled to a catheter. In some embodiments, the user interface can be configured to receive a selection of a total dosage amount, a selection of a length of time over which to administer the total dosage amount, and a particular number of discrete doses out of the total dosage amount to administer during the length of time over which to administer the total dosage amount.

The processor 234 can communicate with one or more of the user interface 236, the battery 162, the communication module 238, the motor 164, the memory 242, the microswitch 188, and the sensors 250. The processor 234 can be configured to receive data from the user interface 236 or from an external device through the communication module 238. Based on the data received from the user interface 236 or external device, the processor can adjust one or more operational parameters of the injection device 100, such as an amount of medicine to dispense or draw into the medicine cartridge 138, activate the motor 164 to drive dispensing of medicine or drawing of medicine into the medicine cartridge 138, prompt the user to input a selection on the user interface 236, store data in the memory 242, and/or access data from the memory 242. Based on the data received from the user interface 236 or external device, the processor can also be configured to adjust an amount of medicine to dispense and an amount of time over which to dispense the medicine in embodiments in which the medicine cartridge 238 is coupled to a catheter. The processor can be configured to activate the motor at one or more times throughout the amount of time over which to dispense medicine to dispense medicine through the catheter. The processor may also be configured to determine rate at which to dispense medicine through the catheter and/or one or more time intervals at which to dispense medicine through the catheter in order to dispense medicine throughout the programmed amount of time over which to dispense medicine.

The processor 234 can also receive data from the microswitch 188, including engagement and disengagement of the microswitch 188. In response to engagement of the microswitch 188 or disengagement of the microswitch 188, the processor 234 can be configured to allow or prevent one or more injection events from occurring. In some embodiments, the processor can be configured to retrieve data from the memory 242, transmit data from the memory 242, the user interface 236, and/or the microswitch 188 to an external device via the communication module 238, and/or display data from the memory 242, the user interface 236, and/or the microswitch 188 via the user interface 236. The processor 234 can further be configured to receive data from the sensors 250. Based on the data received from the sensors 250, the processor can adjust one or more operational parameters of the injection device 100, activate the motor 164 to perform an injection event, prompt the user to input a selection on the user interface 236, store data in the memory 242, access data from the memory 242, display data on the user interface 236, or transmit data to an external device via communication module 238.

The battery 162 is configured to provide power to the injection device 100. The battery 162 can provide power to the processor 234, the user interface 236, the communication module 238, the motor 164, the memory 242, and the microswitch 188. The battery 162 can comprise any electrical power source. The battery 162 may be rechargeable, for example, through charging port 116.

In accordance with an illustrative embodiment, the motor 164 can be a stepper motor. However, the motor 164 can comprise any electric motor capable of operating the gear assembly 166.

The communication module 238 is configured to transmit data to and/or receive data from one or more external devices. The communication module 238 can be configured to communicate with external devices through, for example, wireless communication, Bluetooth communication (BTLE), or cellular communication. The communication module 238 may connect to a personal electronic device such as a smart phone, tablet, or personal computer, or may transmit data to a server. The communication module 238 may communicate information to a user, a physician, a pharmacy, a provider, a materials supplier or any other interested party. The injection device 100 may further be configured to communicate, via the communication module 238, with other medical devices, such as, for example, an analyte measuring device. The injection device 100 may receive information from an analyte measuring device relevant to the time and amount of medicine for an injection event. The injection device 100 can be configured to communicate with one or more external devices as part of a health management system. Examples of such a system are described in U.S. Patent Publication No. 2015/0205930 published on Jul. 23, 2015, and U.S. patent application Ser. No. 14/371,401 filed on Jul. 9, 2014, the content of each being hereby incorporated by reference in their entirety. The communication module 238 can include one or more of a transmitter, a receiver, and a transceiver.

In an illustrative embodiment, an analyte monitoring device may have wireless connectivity and can communicate data to a cloud. The analyte monitoring device may transmit data to the medicine injection device 100 related to the injection, such as a recommended amount or time for injection. The analyte monitoring device also may transmit data related to measurement of analytes, user input data, and the supply stock of analyte measuring materials, such as lancets or glucose test strips in a blood glucose meter. This data can be monitored by a user, a physician, a pharmacy, a provider, and/or a materials supplier. Alternatively, data may be automatically sent to a user, physician, pharmacy, provider, and/or materials supplier. Data can be automatically uploaded by the analyte monitoring device. Other devices may be connected to the cloud, and may communicate with the analyte monitoring device and/or the medicine injection device, such as a computer, a server, a database, a personal electronic device, or a scale.

The sensors 250 can be configured to determine various states and conditions related to the injection device 100. The one or more sensors 250 can include one or more of an electronic caliper, a magnetic sensor, a timer, a barcode scanner or an RF reader. The injection device 100 can include sensors 250 for determining the type of medicine inserted into the device, for example, different types of insulin. The sensors 250 may detect a code on a vessel containing the medicine, such as a bar code or RFID tag. A bar code scanner or RF reader may be used. The sensors 250 can also include sensors for determining a time at which an injection event occurred and an amount of medicine involved in the injection event. The sensors 250 can include a timer for determining a time at which an injection event occurred. The injection device 100 may use information from the timer to notify a user of a recent injection event if the user attempts to initiate injection multiple times in a short time frame. An electronic caliper may be used to determine the amount of medicine injected. In one embodiment, the electronic caliper can measure movement of the shaft 170 of the longitudinal screw nut 168. Alternatively, a magnetic sensor may be used to sense the movement of a magnet on a gear of the device (such as the manual drive gear 176, one or more of the motorized drive gears 180, or a counter wheel gear) to determine the amount of medicine injected. The magnetic sensor may also be used to measure the longitudinal movement of the shaft 170 of the screw nut 168. The sensors 250 may further determine the amount of medicine drawn into the cartridge 138 and the amount of medicine remaining in the cartridge 138 following an injection event. The sensors can be configured to transmit sensor data to the processor 234. The processor 234 can store the sensor data in the memory 242, transmit the sensor data to an external device via communication module 238, display the sensor data on the user interface 236, or change an operational setting of the injection device 100 in response to the sensor data.

The injection device 100 may further include one or more indicators to provide notifications or alerts to a user. For example, the indicators can be configured to alert a user to a time to initiate an injection event, an end of an injection event, an amount of medicine injected, a warning regarding a possible duplicative injection, and/or receipt of data from an external device. The indicators can include visible, audible, and haptic indicators. A visible indicator may include one or more lights, such as an LED. A visible indication may also appear on the user interface 236. An audible indicator can include one or more speakers. A haptic indicator may include a motor engaged to a weight in order to provide vibration. A time for an alert may be programmed into the medicine injection device 100. Alternatively, an alert can be transmitted from an external device, such as an analyte monitoring device, and may be based at least in part on data measured by the analyte testing device.

In some embodiments, an indication is provided to indicate completion of an injection. The indication of completion of an injection can comprise an audible indicatation, such as for example, a beep, chirp, or ringing sound. The indication of completion of an injection may also comprise a haptic indication such as a vibration. In some embodiments, the indication for completion of an injection can comprise a visible indication, such as a flashing of a light.

In some embodiments, an indication is provided to warn a user of a recently performed injection. Such an indication may be provided if a determination is made, via the processor 234, for example, that a user is attempting to initiate a subsequent injection following a recently performed injection. The indication of a recently performed injection can comprise an audible indicatation, such as for example, a beep, chirp, or ringing sound. The indication of a recently performed injection may also comprise a haptic indication such as a vibration. In some embodiments, the indication of a recently performed injection can comprise a visible indication, such as a flashing of a light.

Dispensing Device with Knob-Based Manual Dispensing Actuator

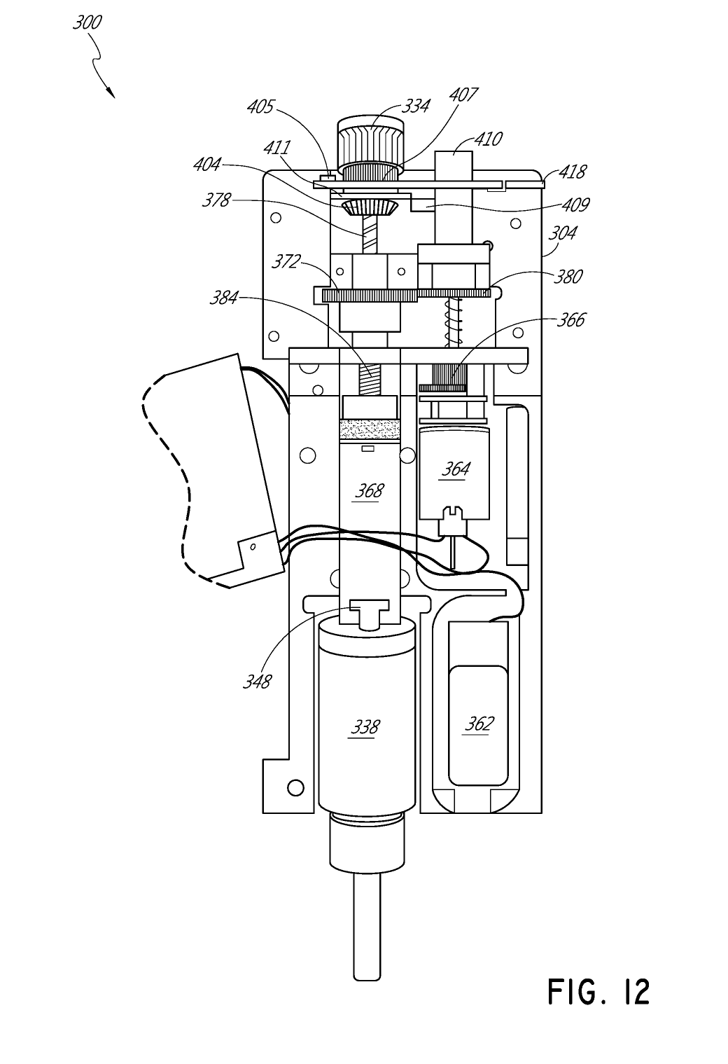

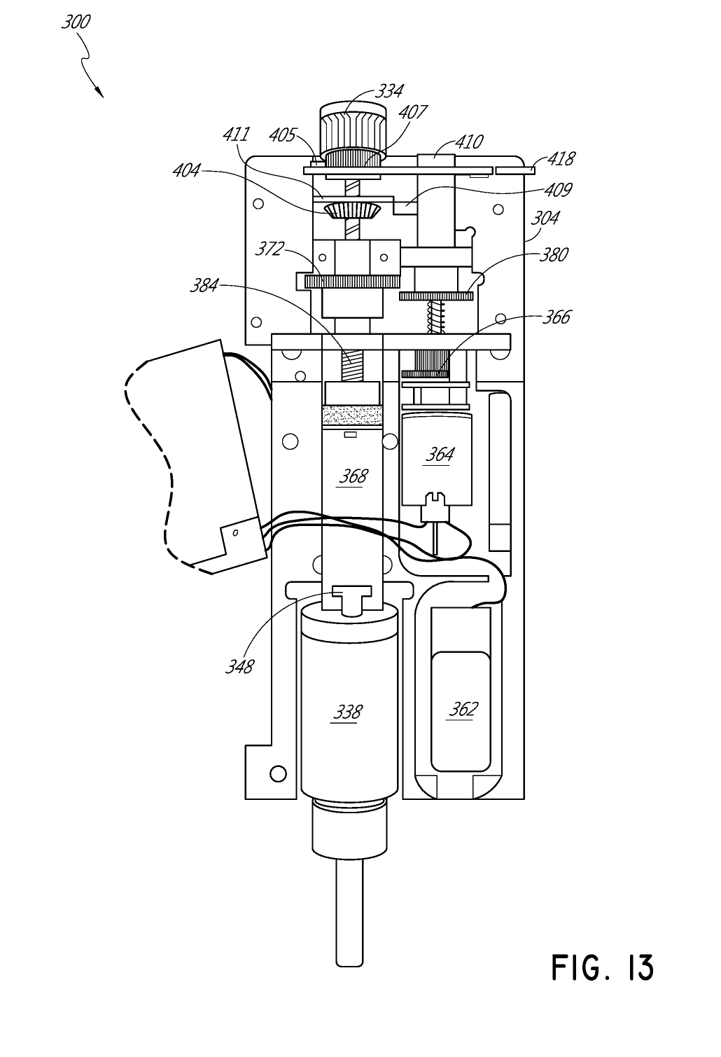

FIG. 12 depicts internal components of an alternative injection device 300 in accordance with an illustrative embodiment of the present invention. The injection device 300 may include similar features to the injection device 100. The injection device 300 includes a body 304, a medicine cartridge 338 positioned within the body 304, a lead screw 384, a screw nut 368, a plunger assembly 348, a motor 364, gear assembly 366, a power module 362, a rotatable manual dispensing knob 334, a manual activation button 410, a counter wheel gear 404, a gear shaft 378, and a reset button 418.

The motor 364 can engage the gear assembly 366. The gear assembly 366 includes a motorized drive gear 380 and a screw drive gear 372. Screw drive gear 372 can be coupled to the lead screw 384. The lead screw 384 can be coupled to screw nut 368. Screw nut 368 can couple to the plunger assembly 348. Rotation of the screw drive gear 372 can cause rotation of the lead screw 384. Rotation of the lead screw 384 can cause longitudinal movement of the screw nut 368 in the direction of a distal end of the injection device 300, which can cause displacement of the plunger assembly 348 towards the distal end of the injection device 300. Displacement of the plunger assembly 348 towards the distal end of the injection device 300 can cause medicine to be dispensed from the cartridge 338. An electronic counter linked to the motor 364 and screw nut 368 movement can control the dispensing. The injection device 300 can further include a reverse control to reset the injection device 300 to accept a new cartridge and allow the injection device 300 to be used for multiple injections. The injection device 300 can also be used to fill the cartridge 338 using a reverse feature, in which the screw drive gear 372 is rotated in a reverse direction from that used to dispense medicine from the cartridge 338. The injection device 300 is configured to allow for both a motorized and manual injection.

### Motorized Injection Mechanism

FIG. 12 shows the injection device 300 in a configuration for operation of the motorized injection mechanism. FIG. 12 shows the motorized drive gear 380 engaged to the screw drive gear 372. The motor 364 is engaged to the gear assembly 366 such that activation of the motor 364 can cause rotation of the motorized drive gear 380. The motor 364 may be activated, and the medicine administered, when a button, trigger, or other activation mechanism on the portable medicine injection device is activated. When the motorized drive gear 380 is engaged to the screw drive gear 372, rotation of the motorized drive gear 380 can cause rotation of the screw drive gear 372. As previously described, rotation of the screw drive gear 372 can cause a dispensing of medicine from the cartridge 338 or filling of the cartridge 338. The manual activation button 410 extends out of the proximal end of the body 304 of the injection device 300.

### Manual Injection Mechanism

The manual activation button 410 can be advanced distally into the body 304 of the injection device 300 in order to distally advance the motorized drive gear 380, such that the motorized drive gear 380 is disengaged from the screw drive gear 372. The manual activation button 410 is further coupled to a platform 409. The counter wheel gear 404 is affixed to a platform 411, and the platform 411 is positioned at least partially on top of a proximal surface of the platform 409, the platform 409 preventing the platform 411 from advancing towards the distal end of the injection device 100. Advancement of the manual activation button 410 may further cause the platform 409 to advance towards the distal end of the injection device 100, allowing the platform 411 to advance towards the distal end of the injection device 100. Advancement of the platform 411 towards the distal end of the injection device 100 causes counter wheel gear 404 to move from a first position in which the counter wheel gear 404 is not coupled to a counter wheel to a second position in which the counter wheel gear 404 is connected to a counter wheel, when a counter wheel is positioned within the injection device 300. In some embodiments, when the motorized drive gear 380 is engaged to the screw drive gear 372, rotation of the dispensing knob 334 may be prevented. If the motorized drive gear 380 is disengaged from the screw drive gear 372, rotation of the dispensing knob 334 can be configured to cause rotation a dispensing knob gear 407, coupled to the dispensing knob 334 and gear shaft 378, which can cause rotation of the gear shaft 378. Advancement of the manual activation button 410 can also cause a pawl 405 to engage the knob gear 407, such that rotation of the dispensing knob 334 is permitted in only a single direction. Rotation of the gear shaft 378 can cause rotation of the counter wheel gear 404 and the screw drive gear 372. As described above, rotation of the screw drive gear 372 can cause medicine to be dispensed from the cartridge 338 or drawn into the cartridge 338. The counter wheel gear 404 can be connected to a counter wheel (not shown). Rotation of the counter wheel gear 404 can cause rotation of the counter wheel. The reset button 418 can be used to reset one or more of the counter wheel and the button 410. The reset button 418 is coupled to the pawl 405. When the motorized drive gear 380 is engaged with the screw drive gear 372, the pawl 405 is disengaged from the dispensing knob gear 407. When the pawl 405 is engaged with the knob gear 407, the pawl 405 may also be configured to allow for rotation of the counter wheel gear in only one direction. Accordingly, disengagement of the pawl 405 from the dispensing knob gear 407 can allow the counter wheel to be reset.

FIG. 13 depicts internal components of an injection device 300 in accordance with an illustrative embodiment of the present invention showing the injection device 300 configured for use of the manual injection mechanism. The motorized drive gear 380 is disengaged from the screw drive gear 372. FIG. 13 further shows the pawl 405 disengaged from the dispensing knob gear 407.

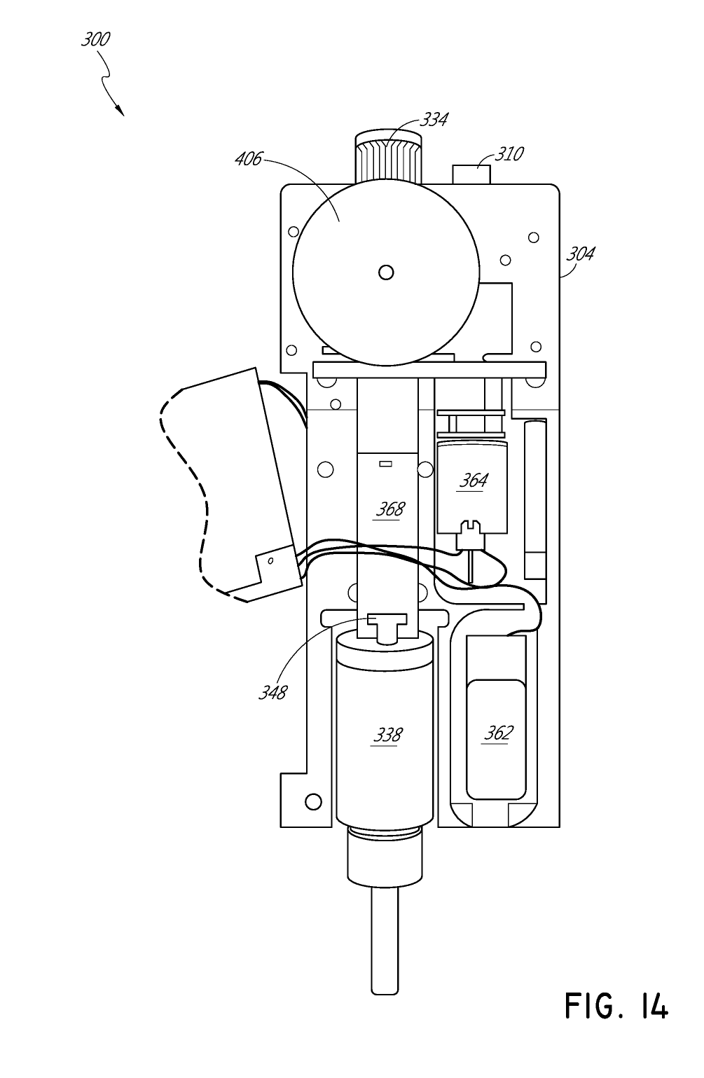

FIG. 14 depicts internal components of an injection device 300 in accordance with an illustrative embodiment of the present invention showing a counter wheel 406 positioned within the injection device 300.

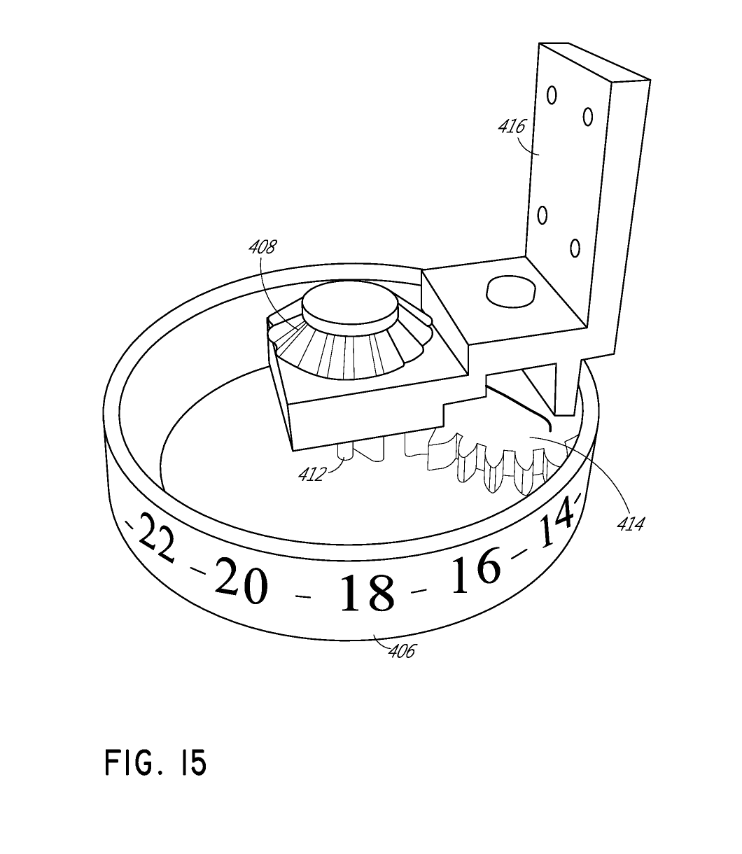

FIG. 15 depicts a counter wheel 406 in accordance with an illustrative embodiment of the present invention. The counter wheel includes a bracket 416, a gear 408, a gear 412, a gear 414 and a numbered edge 413. The bracket 416 is configured to secure the counter wheel to the injection device 300. The gear 408 is configured to engage the counter wheel gear 404, such that rotation of the counter wheel gear 404 causes rotation of the gear 408. The gear 408 is coupled to the gear 412, such that rotation of the gear 408 causes rotation of the gear 412. The gear 412 is engaged with the gear 414, such that rotation of the gear 414 causes rotation of the gear 412. The gear 414 engages an interior surface of the numbered edge 413, such that rotation of the gear 414 cause rotation of the numbered edge 413. The numbered edge 413 includes a plurality of consecutive umbers across the exterior surface of the numbered edge 413 such that rotation of the numbered edge 413 allows for the consecutive numbers to pass through a viewing area visible to a user. The numbered edge 413 may be configured to rotate at a rate such that the number visible in the viewing area corresponds to the amount of medicine dispensed from the medicine injection device. Gear 414 may be spring loaded, so that disengagement of the counter wheel gear 404 causes the counter wheel to return to a starting position, in which the number visible in the viewing area is zero.

Implementations disclosed herein provide systems, methods, and apparatus for a medical injection device. One skilled in the art will recognize that these embodiments may be implements in hardware, software, firmware, or any combination thereof.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. Disk and disc, as used herein, includes compact disc (CD), laser disc, optical disc, digital versatile disc (DVD), floppy disk and Blu-ray® disc where disks usually reproduce data magnetically, while discs reproduce data optically with lasers. It should be noted that a computer-readable medium may be tangible and non-transitory. The term “computer-program product” refers to a computing device or processor in combination with code or instructions (e.g., a “program”) that may be executed, processed or computed by the computing device or processor. As used herein, the term “code” may refer to software, instructions, code or data that is/are executable by a computing device or processor.

Software or instructions may also be transmitted over a transmission medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component or directly connected to the second component. As used herein, the term “plurality” denotes two or more. For example, a plurality of components indicates two or more components.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

In the foregoing description, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures and techniques may be shown in detail to further explain the examples.

Headings are included herein for reference and to aid in locating various sections. These headings are not intended to limit the scope of the concepts described with respect thereto. Such concepts may have applicability throughout the entire specification.

It is also noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. A medicine dispensing device, comprising:
a recess configured to receive a removable medicine cartridge;
a gear assembly;
a first slidable member coupled to the gear assembly and comprising a medicine cartridge plunger coupling member, wherein rotation of the gear assembly is configured to cause longitudinal movement of the first slidable member within the recess;
a motor coupled to the gear assembly, where activation of the motor is configured to cause rotation of the gear assembly; and
a manual dispensing actuator having an engaged and an unengaged position, wherein when in the engaged position, movement of the manual dispensing actuator causes rotation of the gear assembly without activation of the motor, and when in the unengaged position, the manual dispensing actuator is not coupled to the gear assembly.

2. The medicine dispensing device of claim 1, wherein the manual dispensing actuator is biased in the unengaged position.

3. The medicine dispensing device of claim 1, wherein the manual dispensing actuator can be moved to the engaged position without operating any other actuator.

4. The medicine dispensing device of claim 1, wherein the manual dispensing actuator comprises a second slidable member

5. The medicine dispensing device of claim 4, wherein a first position of the second slidable member is the unengaged position, and wherein the second slidable member is configured to slide to a second position that is the engaged position, and wherein further sliding of the second slidable member causes rotation of the gear assembly.

6. The medicine dispensing device of claim 1, comprising a rotatable counter wheel having a plurality of numbers inscribed thereon and a counter wheel opening, wherein the counter wheel opening is configured to provide visibility of one of the plurality of numbers.

7. The medicine dispensing device of claim 6, wherein the counter wheel is coupled to the manual dispensing actuator; wherein actuation of the manual dispensing actuator causes one or more discrete amounts of medicine to be dispensed and causes rotation of the counter wheel such that a number visible in the counter wheel opening is indicative of the number of the one or more discrete amounts of medicine dispensed.

8. The medicine dispensing device of claim 1, wherein the first slidable member comprises an internally threaded shaft and the medicine dispensing device further comprises an elongated screw member threadably engaged with the internally threaded shaft, the elongated screw member coupled to the gear assembly such that rotation of the gear assembly causes rotation of the elongated screw member, thereby causing longitudinal movement of the first slidable member.

9. The medicine dispensing device of claim 1, wherein the gear assembly comprises a manual drive gear and one or more motorized drive gears, the manual drive gear is coupled to one of the motorized drive gears, wherein when in the engaged position, movement of the manual dispensing actuator causes rotation of the manual drive gear, wherein rotation of the manual drive gear causes rotation of the motorized drive gears, wherein rotation of the motorized drive gears causes longitudinal movement of the first slidable member.

10. The medicine dispensing device of claim 1 further comprising:
a user interface, the user interface including a display configured to display data related to the dispensing of medicine from the medicine device, wherein the user interface is further configured to receive a user selection of an amount of medicine; and
a processor configured to receive data from the user interface, wherein the processor is configured to instruct the motor to drive the gear assembly.

11. A medicine dispensing device, comprising:
a recess configured to receive a removable medicine cartridge;
a gear assembly;
a first slidable member coupled to the gear assembly and comprising a medicine cartridge plunger coupling member, wherein rotation of the gear assembly is configured to cause longitudinal movement of the first slidable member within the recess, wherein the medicine cartridge plunger coupling member comprises a slot configured to receive a T-shaped portion of the medicine cartridge plunger assembly, wherein the slot is configured to allow the T-shaped portion of the medicine cartridge plunger assembly to rotate within the slot between a locked and unlocked position, wherein when in the locked position, retraction of the first slidable member retracts the medicine cartridge plunger assembly, and when in the unlocked position, retraction of the first slidable member disengages from and does not retract the medicine cartridge plunger assembly; and
a motor coupled to the gear assembly, where activation of the motor is configured to cause rotation of the gear assembly.

12. The medicine dispensing device of claim 11, wherein the motor is configured to rotate the gear assembly in a forward direction and a reverse direction, wherein rotation of the gear assembly in the forward direction causes the first slidable member to advance into the recess, and wherein rotation of the gear assembly in the reverse direction causes the first slidable member to retract out of the recess.

13. The medicine dispensing device of claim 12, wherein rotation of the gear assembly in the forward direction causes the medicine cartridge plunger assembly to advance towards a dispensing end of the medicine cartridge, wherein advancement of the plunger assembly towards the dispensing end of the medicine cartridge causes fluid between the medicine cartridge plunger assembly and the dispensing end of the medicine cartridge to be dispensed from the medicine cartridge, wherein rotation of the gear assembly in the reverse direction causes the medicine cartridge plunger assembly to retract away from the dispensing end of the medicine cartridge, wherein retraction of the medicine cartridge plunger assembly away from the dispensing end of the medicine cartridge causes the dispensing end of the medicine cartridge to draw fluid in to the medicine cartridge.

14. The medicine dispensing device of claim 11, further comprising:
one or more sensors configured to detect a position of the medicine cartridge within the recess;
one or more sensors configured to detect an injection event; and
a processor configured to limit a number of injection events that can be performed when the medicine cartridge is in an injection position.

15. The medicine dispensing device of claim 14, wherein in response to a detection that the medicine cartridge has moved away from an injection position, the processor is configured to retract the first slidable member, wherein the first slidable member disengages from and does not retract the medicine cartridge plunger assembly.

16. The medicine dispensing device of claim 11, further comprising a removable medicine cartridge within the recess, the removable medicine cartridge comprising:
a chamber configured to store medicine therein;
a plunger assembly positioned within the chamber, configured to move longitudinally within the chamber, wherein the plunger assembly comprises a plunger seal and a plunger insert, wherein the plunger insert comprises a T-shaped portion coupled to the medicine cartridge plunger coupling member; and
a septum configured to receive a needle.

17. The medicine dispensing device of claim 16, wherein the removable medicine cartridge comprises a tab extending from an exterior wall of the removable medicine cartridge, wherein the tab is configured to allow for rotation of the removable medicine cartridge within the recess, wherein rotation of the removable medicine cartridge within the recess allows for coupling and uncoupling of the removable medicine cartridge with the first sliding member.

18. The medicine dispensing device of claim 17, further comprising a switch extending at least partially into the recess configured to detect the presence of the cartridge within the recess.

19. The medicine dispensing device of claim 11, further comprising a medicine cartridge comprising:
a chamber configured to store medicine therein;
a plunger assembly positioned within the chamber, configured to move longitudinally within the chamber, wherein the plunger assembly comprises a plunger seal and a plunger insert, wherein the plunger insert is configured to couple with a medicine cartridge plunger coupling member of a medicine dispensing device;
a tab extending from an exterior wall of the medicine cartridge, wherein the tab is configured to allow for rotation of the removable medicine cartridge within a recess of a medicine dispensing device, wherein rotation of the removable medicine cartridge within the recess allows for coupling and uncoupling of the removable medicine cartridge with the medicine cartridge plunger coupling member;
a ridge extending from the exterior wall of the medicine cartridge, wherein the ridge is positioned to allow for activation of a switch in the interior of the medicine dispensing device; and
a septum configured to receive a needle.

20. The medicine dispensing device of claim 11, further comprising
a removable medicine cartridge within the recess, the removable medicine cartridge comprising:
a chamber configured to store medicine therein; and
a plunger assembly positioned within the chamber, configured to move longitudinally within the chamber, wherein the plunger assembly comprises a plunger seal and a plunger insert, wherein the plunger insert comprises a T-shaped portion coupled to the medicine cartridge plunger coupling member;
a catheter secured to a dispensing end of the medicine cartridge, wherein the catheter is configured to receive medicine dispensed from the medicine cartridge.

21. The medicine dispensing device of claim 20, further comprising a processor, wherein the processor is configured to:
receive instructions for an amount of medicine to dispense from the medicine cartridge through the catheter and a length of time over which to dispense the amount of medicine; and
activate the motor based on the instructions for an amount of medicine to dispense from the medicine cartridge through the catheter and a length of time over which to dispense the amount of medicine.

22. The medicine dispensing device of claim 11, further comprising a motorized dispensing actuator, wherein the motorized dispensing actuator is configured to actuate the motor while an external pressure is being applied to the motorized dispensing actuator.

23. A kit, comprising:
a medicine dispensing device, comprising:
a recess configured to receive a removable medicine cartridge;
a gear assembly;
a first slidable member coupled to the gear assembly and comprising a medicine cartridge plunger coupling member, wherein rotation of the gear assembly is configured to cause longitudinal movement of the first slidable member within the recess; and
a motor coupled to the gear assembly, where activation of the motor is configured to cause rotation of the gear assembly; and
a medicine filling tool, wherein the medicine filling tool comprises:
an adapter member configured to couple to an end of the medicine dispensing device; and
a hollow cylinder coupled to the adapter member and configured to receive a medicine vial, wherein the filling tool is shaped such that placement of a medicine vial within the hollow cylinder aligns the medicine vial with the recess and any medicine cartridge therein.

24. The kit of claim 23, further comprising a removable medicine cartridge within the recess, the removable medicine cartridge comprising:
a chamber configured to store medicine therein;
a plunger assembly positioned within the chamber, configured to move longitudinally within the chamber, wherein the plunger assembly is coupled to the medicine cartridge plunger coupling member; and
a septum configured to receive a needle.

25. A method for operating a medicine dispensing device, comprising:
inserting a medicine cartridge into a recess of a medicine dispensing device, the medicine dispensing device comprising:
a recess configured to receive a removable medicine cartridge;
a gear assembly;
a first slidable member coupled to the gear assembly and comprising a medicine cartridge plunger coupling member, wherein rotation of the gear assembly is configured to cause longitudinal movement of the first slidable member within the recess; and
a motor coupled to the gear assembly, where activation of the motor is configured to cause rotation of the gear assembly;
the medicine cartridge comprising:
a chamber configured to store medicine therein;
a plunger assembly positioned within the chamber, configured to be moveable longitudinally within the chamber; and
a septum at one end of the chamber;
inserting one end of a needle assembly into the septum;
inserting an opposite end of the needle assembly into a medicine vial containing medicine; and
drawing medicine from the medicine vial through the needle assembly into the chamber of the medicine cartridge;
dispensing medicine from the medicine cartridge through the needle.

26. The method of claim 25, further comprising activating the motor to perform one or more of drawing medicine from the medicine vial and dispensing medicine from the medicine cartridge.

27. The method of claim 26, further comprising programming one or more of an amount of medicine to draw from the medicine vial and an amount of medicine to dispense from the medicine cartridge using a user interface.

28. The method of claim 25, further comprising detecting, via one or more sensors, the occurrence of an injection event.

29. The method of claim 25, further comprising:
positioning the medicine cartridge into an injection position, wherein the medicine dispensing device is configured so that an injection event is prevented if the medicine cartridge is not in the injection position;
detecting, via one or more sensors, the presence of the medicine cartridge in the injection position;
detecting, via one or more sensors, the occurrence of one or more injection events; and
limiting the number of injection events that can occur when the medicine cartridge is in the injection position.

30. The method of claim 25, further comprising:
coupling the plunger assembly of the medicine cartridge with the plunger coupling member of the medicine dispensing device, wherein dispensing medicine comprises advancing the plunger assembly within the interior of the medicine cartridge, wherein the advancing the plunger assembly comprises advancing the plunger coupling member of the medicine dispensing device; and
moving the medicine cartridge out of the injection position, wherein in response to moving the medicine cartridge out of the injection position, the plunger coupling member uncouples from the plunger assembly and automatically retracts without retracting the plunger assembly.
